(19) United States
(12) Patent Application Publication
(10) Pub. No.: US 2022/0364173 A1
(43) Pub. Date: Nov. 17, 2022

(54) METHODS AND SYSTEMS FOR DETECTION OF NUCLEIC ACID MODIFICATIONS

(71) Applicant: The University of Chicago, Chicago, IL (US)

(72) Inventors: Chuan HE, Chicago, IL (US); Lulu HU, Chicago, IL (US)

(73) Assignee: The University of Chicago, Chicago, IL (US)

(21) Appl. No.: 17/754,622

(22) PCT Filed: Oct. 9, 2020
(86) PCT No.: PCT/US2020/070639

§ 371 Date: Apr. 7, 2022

Related U.S. Application Data
(60) Provisional application No. 62/913,475, filed on Oct. 10, 2019.

(51) Int. Cl. C12Q 1/6883

(52) U.S. Cl. CPC C12Q 1/6883; C12Q 2600/154; C12Q 2600/158; C12Q 1/6869

(57) ABSTRACT

Aspects of the present disclosure relate to methods for modification and detection of methylated nucleotides. Embodiments are directed to detection of RNA methylation. Disclosed are methods and compositions for transcriptome-wide detection of N⁶-methyladenosine in mRNA. In some cases, methods for modifying a methylated nitrogenous base are described. Also disclosed are enzymes and other molecules useful for RNA methylation detection.

### CROSS-REFERENCE TO RELATED APPLICATIONS

This application claims the benefit of priority of U.S. Provisional Patent Application No. 62/913,475 filed Oct. 10, 2019, which is hereby incorporated by reference in its entirety.

### STATEMENT OF GOVERNMENT RIGHTS

This invention was made with government support under HG008935 awarded by the National Institutes of Health. The government has certain rights in the invention.

### BACKGROUND OF THE INVENTION

### Field of the Invention

This invention relates generally to the field of molecular biology. Certain aspects relate to methods and compositions for detection of methylated nucleic acid molecules.

### II. BACKGROUND

Nucleic acids carry a wide range of chemical modifications. Many of these modifications are used to exert essential influences on a variety of cellular and biological processes. RNA modifications have recently emerged as critical posttranscriptional regulators of gene expression programs. They affect diverse eukaryotic biological processes, and the correct deposition of many of these modifications is required for normal development¹. RNA modifications are integral to the regulation of RNA metabolism. The most abundant internal mRNA modification is N⁶-methyladenosine (m⁶A), which affects almost all the aspects of RNA metabolism, including splicing, translation and degradation². However, the mechanistic roles of m⁶A in different developmental processes and biological contexts still remain elusive.

New tools that can delineate the transcriptome-wide distribution of m⁶A at nucleotide resolution with the critical modification fraction information at each modified site are needed to understand biological relevance and impacts of the modified transcripts and sites. Recognized herein is a need for accurate, high-throughput methods and compositions for detection of nucleic acid modification, including RNA modifications such as N⁶-methyladenosine.

### SUMMARY OF THE DISCLOSURE

The current disclosure fulfils the need in the art for methods and compositions for detection of nucleic acid modifications, such as N⁶-methyladenosine. Accordingly, certain aspects of the disclosure relate to methods for detecting N⁶-methyladenosine in mRNA. Embodiments relate to methods for modifying a nitrogenous base methylated at a nitrogen atom. For example, certain embodiments are directed to methods for attaching a functional group to a methylated nitrogen on an adenosine base using a dimethyltransferase enzyme. Example compositions useful in the disclosed methods include S-adenosyl-1-methionine (SAM) analogs. Further embodiments are directed to natural or engineered enzymes useful in N⁶-methyladenosine detection.

In some embodiments, disclosed herein are methods for detecting a methylated nucleotide, methods for analyzing a methylated nucleotide, methods for analyzing a nucleic acid molecule, methods for analyzing a messenger ribonucleic acid molecule, methods for analyzing a deoxyribonucleic acid molecule, methods for modifying a nitrogenous base, methods for modifying a methylated nitrogenous base, methods for attaching a functional group to a methylated nucleotide, methods for transcriptome analysis, methods for analyzing RNA methylation of a transcriptome, methods for identifying a nucleotide as methylated, methods for identifying an adenosine as methylated at an N⁶ nitrogen atom, methods for methylome analysis, methods for detecting a condition associated with nucleic acid methylation in an individual, methods for generating an engineered enzyme, and methods for directed evolution of a methyltransferase. It is contemplated that any one or more of these embodiments may be excluded from embodiments of the present disclosure.

The methods of the disclosure may include 1, 2, 3, 4, 5, 6, 7, 8, 9, 10, 11, 12, 13, 14, or 15 or more of the following steps which may be performed in any order and repeated throughout any specific method embodiments: obtaining nucleic acid molecules; obtaining nucleic acid molecules from a biological sample; obtaining a biological sample containing nucleic acids from a subject; isolating nucleic acid molecules; purifying nucleic acid molecules; obtaining an array or microarray containing nucleic acids to be modified; denaturing nucleic acid molecules; shearing or cutting nucleic acid molecules; hybridizing nucleic acid molecules; fragmenting nucleic acids; incubating a nucleic acid molecule with an enzyme; incubating a nucleic acid molecule with a ligase; incubating a nucleic acid molecule with a nuclease; incubating a nucleic acid with a methyltransferase; incubating a nucleic acid molecule with a diatomic halogen molecule; incubating a nucleic acid molecule with 12, incubating a nucleic acid molecule with a restriction enzyme; attaching one or more functional groups to a nucleic acid; attaching one or more functional groups to a methylated nucleotide; attaching one or more functional groups to a nitrogenous base methylated at a nitrogen atom; subjecting an RNA molecule to reverse transcription; amplifying a nucleic acid molecule, sequencing a nucleic acid molecule, identifying a methylated nucleotide in a nucleic acid molecule based on a sequence; and generating a complementary nucleic acid molecule from an RNA molecule. It is contemplated that any one or more of these steps may be excluded from a method of the present disclosure.

It is contemplated that some embodiments will involve steps that are done in vitro, such as by a person or a person controlling or using machinery to perform one or more steps.

In other methods, there may be steps including, but not limited to, obtaining information (qualitative and/or quantitative) about one or more adenosine modifications in a nucleic acid sample; ordering an assay to determine, identify, and/or map adenosine modifications in a nucleic acid sample; reporting information (qualitative and/or quantitative) about one or more adenosine modifications in a nucleic acid sample; comparing that information to information about a different adenosine modification in a control or comparative sample. Unless otherwise stated, the terms “determine,” “analyze,” “assay,” and “evaluate” in the context of a sample refer to chemical or physical transformation of that sample to gather qualitative and/or quantitative data about the sample. Moreover, the term “map” means to identify the location within a nucleic acid sequence of the particular nucleotide.

Compositions or kits of the present disclosure can include one or more of the following: a nucleic acid, a natural enzyme, an engineered enzyme, a polymerase, a ligase, a reverse transcriptase, a methyltransferase, a dimethyltransferase, an RNA demethylase, a S-adenosyl-1-methionine analog, a primer, and deoxynucleoside triphosphates (dNTPs). Any one or more components may be excluded from compositions or kits of the present disclosure.

As used herein, a “S-adenosyl-1-methionine analog” or “SAM analog” describes a molecule which was derived or generated from S-adenosyl-1-methionine, for example by removal, addition, or substitution of one or more chemical moieties, or which is a chemical or structural analog of S-adenosyl-1-methionine. A SAM analog may be a molecule which is identical in structure to S-adenosyl-1-methionine with the exception of one or more chemical moieties. For example, a SAM analog may be identical in structure to S-adenosyl-1-methionine but for the methyl group attached to the sulfur atom, which is instead a different chemical moiety (e.g., a functional group such as an allyl group). Various SAM derivatives are described herein and include, for example, allyl-SAM.

In some embodiments, nucleic acid molecules analyzed or modified by the disclosed methods may be DNA, RNA, or a combination of both. Nucleic acids may be recombinant, genomic, or synthesized. In additional embodiments, methods involve nucleic acid molecules that are isolated and/or purified. In some embodiments, the nucleic acid molecules are fragmented. In some embodiments, the nucleic acid molecules are natural fragments. Natural fragments refers to nucleic acid molecules that exist in nature as fragments, such as cell-free DNA and cell-free RNA, by way of example. The nucleic acid may be isolated from a cell or biological sample in some embodiments. Certain embodiments involve isolating nucleic acids from a eukaryotic, mammalian, or human cell. In some cases, nucleic acids are separated or isolated from non-nucleic acids. In some embodiments, the nucleic acid molecule is eukaryotic; in some cases, the nucleic acid is mammalian, which may be human. In these embodiments, the nucleic acid molecule is isolated from a human cell and/or has a sequence that identifies it as human. In particular embodiments, it is contemplated that the nucleic acid molecule is not a prokaryotic nucleic acid, such as a bacterial nucleic acid molecule. In some cases, a nucleic acid is isolated by any technique known to those of skill in the art, including, but not limited to, using a gel, column, matrix or filter to isolate the nucleic acids. In some embodiments, the gel is a polyacrylamide or agarose gel.

Disclosed herein, in some embodiments, is a method for detecting a methylated nucleotide of a nucleic acid molecule comprising (a) incubating the nucleic acid molecule with a methyltransferase enzyme and a S-adenosyl-1-methionine (SAM) analog comprising a functional group under conditions sufficient to attach the functional group to the methylated nucleotide; (b) subjecting the nucleic acid molecule to conditions sufficient to generate a complementary nucleic acid molecule comprising a mutation at a residue corresponding to the methylated nucleotide; and (c) sequencing the complementary nucleic acid molecule. In some embodiments, the methylated nucleotide is a methylated adenosine.

Disclosed herein, in some embodiments, is a method for modifying a nitrogenous base methylated at a nitrogen atom comprising: (a) providing a methyltransferase enzyme and a S-adenosyl-1-methionine (SAM) analog comprising a functional group; and (b) subjecting the methyltransferase enzyme and the SAM analog to conditions sufficient to attach the functional group to the nitrogen atom. In some embodiments, the nitrogenous base is a nitrogenous base of a nucleoside. In some embodiments, the nitrogenous base is a nitrogenous base of a nucleotide. In some embodiments, the nucleotide is a nucleotide of a ribonucleic acid (RNA). In some embodiments, the nucleotide is a methylated adenosine. In some embodiments, the nucleotide is N⁶-methyladenosine.

Disclosed herein, in some embodiments, is a method for detecting a methylated nucleotide in a ribonucleic acid comprising: (a) attaching a functional group to a nitrogen atom on the nucleotide; (b) generating, from the ribonucleic acid, a complementary nucleic acid comprising a mutation at a residue corresponding to the nucleotide; and (c) sequencing the complementary nucleic acid. In some embodiments, the nucleotide is a methylated adenosine. In some embodiments, the nucleotide is N⁶-methyladenosine. In some embodiments, (a) comprises providing a S-adenosyl-1-methionine (SAM) analog comprising the functional group.

In some embodiments, the functional group has at least two carbons. In some embodiments, the functional group is an alkyl group having at least two carbons or an olefinic group having at least two carbons. In some embodiments, the functional group is not a methyl group. In some embodiments, the functional group is an allyl group. In some embodiments, the functional group is attached to a sulfur atom of the SAM analog. In some embodiments, the SAM analog has formula:

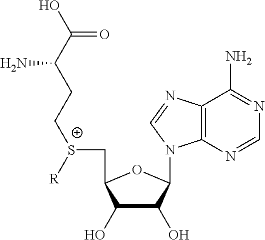

wherein R comprises the functional group. In some embodiments, the SAM analog has formula:

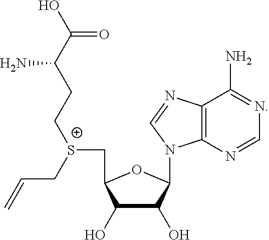

In some embodiments, the methyltransferase is capable of preferentially attaching the functional group to a methylated nucleotide relative to an unmethylated nucleotide under appropriate conditions. In some embodiments, the methyltransferase is an RNA methyltransferase. In some embodiments, the RNA methyltransferase is a dimethyltransferase. In some embodiments, the dimethyltransferase is a Dim1/KsgA dimethyltransferase. In some embodiments, the dimethyltransferase is Dim1 or KsgA. In some embodiments, the dimethyltransferase is HsDim1, ScDim1, or MjDim1. In some embodiments, the dimethyltransferase is MjDim1.

In some embodiments, the method further comprises incubating the nucleic acid molecule or nitrogenous base with a diatomic halogen molecule. In some embodiments, incubating the nucleic acid molecule or nitrogenous base with the diatomic halogen molecule attaches a halogen atom from the diatomic halogen molecule to the nucleic acid molecule or nitrogenous base. In some embodiments, the diatomic halogen molecule is iodine (I₂).

In some embodiments, the method further comprises subjecting the nucleic acid molecule to a reverse transcription reaction with a reverse transcriptase (RT) to generate the complementary nucleic acid molecule. In some embodiments, the complementary nucleic acid molecule is a cDNA molecule. In some embodiments, the RT is any RT suitable for performing reverse transcription. In some embodiments, the RT is an HIV RT or variant thereof, an M-MuLV RT or variant thereof, an AMV RT or variant thereof, a Bst polymerase (e.g., Bst, Bst 2.0, or Bst 3.0) or variant thereof, or a Klentaq polymerase or variant thereof. In some embodiments, the RT is an HIV RT. In some embodiments, the RT is a Bst polymerase or functional fragment thereof. In some embodiments, the RT is Bst 2.0 DNA polymerase. In some embodiments, the polymerase is a Klentaq polymerase or functional fragment thereof.

In some embodiments, the sequencing comprises next generation sequencing. In some embodiments, the sequencing comprises nanopore sequencing. In some embodiments, the methylated nucleotide methylated nucleotide is a methylated adenosine, and the corresponding residue on the complementary nucleic acid does not comprise an adenine. In some embodiments, the methylated nucleotide methylated nucleotide is a methylated adenosine, and the corresponding residue on the complementary nucleic acid comprises a guanine, a thymine, or a cytosine. In some embodiments, the method further comprises identifying the mutation in the complementary nucleic acid as corresponding to the methylated nucleotide. In some embodiments, the nucleic acid molecule is a ribonucleic acid (RNA) molecule. In some embodiments, the ribonucleic acid molecule is a messenger RNA (mRNA).

In some embodiments, the method further comprises providing an oligo-dT primer to the mRNA molecule to generate a double stranded region. In some embodiments, the method further comprises providing a nuclease and subjecting the mRNA to conditions sufficient to digest the double stranded region with the nuclease. In some embodiments, the nuclease is RNase H. In some embodiments, the nucleic acid molecule is a fragment of a longer nucleic acid. In some embodiments, the fragment is between 100 and 200 nucleotides in length. In some embodiments, the nucleic acid molecule is isolated form a sample of a subject. In some embodiments, the nucleic acid molecule is isolated from a biopsy sample. In some embodiments, the sample is a liquid sample. In some embodiments, the nucleic acid molecule is from a vesicle. In some embodiments, the vesicle is an exosome. In some embodiments, the nucleic acid molecule is a cell free nucleic acid molecule. In some embodiments, the cell free nucleic acid molecule is a cell free RNA (cfRNA) molecule.

Disclosed herein, in some embodiments, is a method for analyzing a methylated messenger ribonucleic acid (mRNA) molecule comprising an N⁶-methyladenosine, the method comprising (a) fragmenting the mRNA molecule to generate a fragment comprising the N⁶-methyladenosine; (b) providing a methyltransferase and a S-adenosyl-1-methionine (SAM) analog comprising an allyl group under conditions sufficient to attach the allyl group to the N⁶-methyladenosine in the fragment; (c) incubating the fragment with a reverse transcriptase under conditions sufficient to generate a cDNA molecule comprising a residue corresponding to the N⁶-methyladenosine, wherein the residue comprises a guanine, a thymine, or a cytosine; (d) sequencing the cDNA molecule; and (e) identifying a location of the N⁶-methyladenosine in the mRNA molecule using the sequence. In some embodiments, the method further comprises, prior to (a), incubating the mRNA molecule with an oligo-dT primer under conditions sufficient to hybridize the oligo-dT primer to a complementary region of the mRNA molecule, thereby generating a double stranded region. In some embodiments, the method further comprises providing a nuclease under conditions sufficient to digest the double stranded region. In some embodiments, the nuclease is RNase H. In some embodiments, the SAM analog has formula:

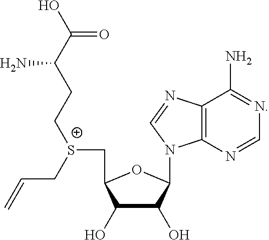

In some embodiments, the methyltransferase is capable of preferentially attaching the functional group to a methylated nucleotide relative to an unmethylated nucleotide under appropriate conditions. In some embodiments, the methyltransferase is an RNA methyltransferase. In some embodiments, the RNA methyltransferase is a dimethyltransferase. In some embodiments, the dimethyltransferase is a Dim1/KsgA dimethyltransferase. In some embodiments, the dimethyltransferase is Dim1 or KsgA. In some embodiments, the dimethyltransferase is HsDim1, ScDim1, or MjDim1. In some embodiments, the dimethyltransferase is MjDim1. In some embodiments, the method further comprises, subsequent to (d), incubating the mRNA molecule with a diatomic halogen molecule. In some embodiments, incubating the mRNA molecule with the diatomic halogen molecule attaches a halogen atom from the diatomic halogen molecule to the nucleotide. In some embodiments, the diatomic halogen molecule is iodine (I₂). In some embodiments, the reverse transcriptase (RT) is any RT suitable for performing reverse transcription. In some embodiments, the RT is an HIV RT or variant thereof, an M-MuLV RT or variant thereof, an AMV RT or variant thereof, a Bst polymerase (e.g., Bst, Bst 2.0, or Bst 3.0) or variant thereof, or a Klentaq polymerase or variant thereof. In some embodiments, the RT is an HIV RT. In some embodiments, the RT is a Bst polymerase or functional fragment thereof. In some embodiments, the RT is Bst 2.0 DNA polymerase. In some embodiments, the polymerase is a Klentaq polymerase or functional fragment thereof. In some embodiments, the mRNA fragment is between 100 and 200 nucleotides in length. In some embodiments, the mRNA molecule is isolated from a sample from a subject. In some embodiments, the mRNA molecule is isolated from a biopsy sample. In some embodiments, the sample is a liquid sample. In some embodiments, the mRNA molecule is isolated from a vesicle. In some embodiments, the vesicle is an exosome. In some embodiments, the mRNA molecule is a cell free ribonucleic acid (cfRNA) molecule.

Embodiments also concern kits, which may be in a suitable container, that can be used to achieve the disclosed methods. Embodiments of the disclosure relate to a kit comprising (a) a SAM analog comprising a functional group and (b) a dimethyltransferase. In some embodiments, the methyltransferase is capable of preferentially attaching the functional group to a methylated nucleotide relative to an unmethylated nucleotide under appropriate conditions. In some embodiments, the methyltransferase is an RNA methyltransferase. In some embodiments, the RNA methyltransferase is a dimethyltransferase. In some embodiments, the dimethyltransferase is a Dim1/KsgA dimethyltransferase. In some embodiments, the dimethyltransferase is Dim1 or KsgA. In some embodiments, the dimethyltransferase is HsDim1, ScDim1, or MjDim1. In some embodiments, the dimethyltransferase is MjDim1. In some embodiments, the functional group has at least two carbons. In some embodiments, the functional group is an alkyl group having at least two carbons or an olefinic group having at least two carbons. In some embodiments, the functional group is not a methyl group. In some embodiments, the functional group is an allyl group. In some embodiments, the functional group is attached to a sulfur atom of the SAM analog. In some embodiments, the SAM analog has formula:

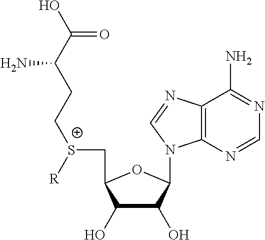

wherein R comprises the functional group. In some embodiments, the SAM analog has formula:

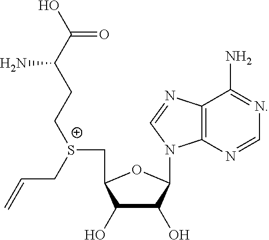

In some embodiments, a kit of the present disclosure further comprises an oligo-dT primer. In some embodiments, the kit comprises a nuclease. In some embodiments, the nuclease is RNase H. In some embodiments, the kit comprises a reverse transcriptase (RT). In some embodiments, the RT is any RT suitable for performing reverse transcription. In some embodiments, the RT is an HIV RT or variant thereof, an M-MuLV RT or variant thereof, an AMV RT or variant thereof, a Bst polymerase (e.g., Bst, Bst 2.0, or Bst 3.0) or variant thereof, or a Klentaq polymerase or variant thereof. In some embodiments, the RT is an HIV RT. In some embodiments, the RT is a Bst polymerase or functional fragment thereof. In some embodiments, the RT is Bst 2.0 DNA polymerase. In some embodiments, the polymerase is a Klentaq polymerase or functional fragment thereof. In some embodiments, the kit further comprises an RNA demethylase. In some embodiments, the RNA demethylase is fat mass and obesity-associated protein (FTO). In some embodiments, the kit further comprises a manganese salt. In some embodiments, the kit further comprises one or more dNTPs. In some embodiments, the kit further comprises nuclease-free water.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description of the Embodiments, Claims, and description of Figure Legends.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A shows a schematic representation of the conversion of m⁶A to allylic-m⁶A and ethanoadenine m⁶A, and of the generation of a mutation at a corresponding residue. FIG. 1B shows a MALDI based mass spectrometry characterization of a m⁶A-containing 12mer template RNA. FIG. 1C shows a MALDI based mass spectrometry characterization of a 12mer template RNA that does not contain any m⁶A. FIG. 1D shows the steady-state kinetics of Mjdim1-catalyzed am⁶A containing and a⁶A containing probes.

FIG. 2 shows a schematic representation of an example m⁶A-sac-seq process.

FIGS. 3A-3F show the results of experiments described in Example 2, including mutation rates (FIG. 3A) and correlation with m⁶A quantity (FIG. 3B), Mjdim1 sequence selectivity (FIG. 3C), mutation ratios for different m⁶A consensus motifs (FIG. 3D), and mismatch proportions for am⁶A vs a⁶A containing probes (FIGS. 3E and 3F).

FIG. 4 shows a flowchart outlining a bioinformatics workflow process for m⁶A-sac-seq analysis.

FIGS. 5A-5D show the results of experiments describes in Example 3, including an overview of identified m⁶A sites (FIG. 5A), metagene profiles (FIG. 5B), m⁶A enrichment (FIG. 5C), and m⁶A distribution (FIG. 5D).

FIGS. 6A and 6B show the results of m⁶A-sac-seq validation using a SELECT method. FIG. 6A shows real-time fluorescence amplification curves and bar plots of Ct values for each target. FIG. 6B shows polyacrylamide gel electrophoresis (PAGE) results for each target.

FIGS. 7A-7C show the results of experiments describes in Example 4. FIG. 7A shows a DNA gel stained with SYBR® Gold nucleic acid gel stain demonstrating readthrough efficiency of wild-type Klentaq enzyme in reverse transcription of an am⁶A-containing template and an a⁶A-containing template. FIG. 7B shows base composition results for cDNA obtained from the am⁶A-containing template and a⁶A-containing template. FIG. 7C shows a DNA gel stained with SYBR® Gold nucleic acid gel stain of cDNA obtained following reverse transcription with wild-type Klentaq enzyme with or without Mn²⁺.

FIG. 8 shows a schematic representation of the process of directed evolution of a Klentaq enzyme using a Broccoli selection platform.

FIG. 9 shows a schematic representation of an example m⁶A-sac-seq method using a modified Klentaq enzyme.

FIGS. 10A and 10B show the results of experiments described in Example 7. FIG. 10A shows a DNA gel stained with SYBR® Gold nucleic acid gel stain demonstrating readthrough efficiency of Bst 2.0 enzyme. FIG. 10B shows base composition results for cDNA obtained from cyclized am⁶A-containing template and a⁶A-containing template.

FIG. 11 shows a schematic representation of an example m⁶A-sac-seq method using a Bst enzyme.

FIGS. 12A-12C show the results of experiments described in Example 8. FIG. 12A shows selected mutation ratio for DRACH motifs. A 53-mer RNA probe with 100% pre-methylated NNm⁶ANN was analyzed by m⁶A-SAC-seq. FIG. 12B shows correlation of mutation ratio versus m⁶A fraction. A set of 53-mers with 0% to 100% pre-methylation level on a GGACU motif was used. Lines represent linear regression. Cross-marks represents data points. FIG. 12C shows mutation patterns for all possible NNm⁶ANN motifs. Each vertical bar represents one motif. The height of the bar represents mutation ratio, respectively (0-100%). “m⁶A probes” are RNA probes containing NNm⁶ANN; “FTO treated” are m⁶A probes treated with FTO to remove most m⁶A; “am⁶A probes” are probes with the allyl group synthetically installed onto m⁶A in RNA probes that contain NNam⁶ANN.

FIG. 13 shows a schematic demonstrating generating a mutation in a cDNA molecule obtained from reverse transcription of a template RNA molecule.

### DETAILED DESCRIPTION OF THE INVENTION

Aspects of this disclosure relate to a method termed m⁶A selective allyl chemical labeling and sequencing, or m⁶A-sac-seq (also “m⁶a-SAC-seq”), with which ribonucleic acid methylation can be identified and quantified at a whole-transcriptome level.

### I. Nitrogenous Base Modification

In certain embodiments, methods involve modification of one or more nitrogenous bases. A “nitrogenous base” describes a molecule which may be associated with a sugar moiety to form a nucleoside or nucleotide and which may be incorporated into a polynucleotide. Nitrogenous bases may be natural, modified, or synthetic. Example nitrogenous bases which may be modified using methods of the present disclosure include adenine (“A”), guanine (“G”), thymine (“T”), cytosine (“C”), uracil (“U”), and variants thereof. In some embodiments, a nitrogenous base is an adenosine. In some embodiments, a nitrogenous base is methylated at a nitrogen atom. A nitrogenous base may be a component of a nucleoside, nucleotide, and/or nucleic acid (e.g., ribonucleic acid, deoxyribonucleic acid, etc.). In some embodiments, a nitrogenous base is a component of N⁶-methyladenosine. The disclosed methods may involve modification of 1, 2, 3, 4, 5, 6, 7, 8, 9, 10, 11, 12, 13, 14, 15, 16, 17, 18, 19, 20, or more nitrogenous bases, or any range derivable therein, per nucleic acid molecule.

Modification may comprise addition of one or more functional groups. In some cases, nitrogenous base modification comprises attachment of a functional group to a nitrogen atom of the nitrogenous base. In some embodiments, the nitrogen atom is a methylated nitrogen atom (e.g., a methylated N⁶ atom of a methylated adenosine). Nitrogenous base modification may modify a nucleotide of a nucleic acid, for example a ribonucleic acid, such that amplification and/or reverse transcription of the nucleic acid results in generation of a mutation corresponding to the nucleotide.

Modification of a nitrogenous base may comprise attachment of a functional group to a methylated nitrogen atom. For example, in some embodiments, a functional group is attached to a methylated N⁶ atom of a methylated adenosine. In some embodiments, the functional group is not a methyl group. In some embodiments, the functional group has at least two carbon atoms. In some embodiments, the functional group is an alkyl group. In some embodiments, the functional group is an olefinic group. In some embodiments, the functional group comprises an alkyne. In some embodiments, the functional group is an allyl group. A functional group may be transferred to a nitrogenous base from a S-adenosyl-1-methionine (SAM) analog. Example SAM analogs are described elsewhere herein and include, for example, allyl-SAM.

A. Modification of Methylated Adenosine

Aspects of the present disclosure relate to modification of a methylated adenosine. In some embodiments, disclosed herein are methods for modification of an N⁶-methyladenosine. N⁶-methyladenosine modification may be useful in detection or identification of N⁶-methyladenosine in a nucleic acid (e.g., mRNA).

In some embodiments, modification of an N⁶-methyladenosine comprises incubating the N⁶-methyladenosine with a methyltransferase enzyme and a SAM analog comprising a functional group under conditions sufficient to attach the functional group to the methylated nitrogen of the N⁶-methyladenosine. Example functional groups are provided herein and include, for example, an allyl group. Example methyltransferase enzymes are provided herein and include, for example, dimethyltransferases such as Dim1/KsgA dimethyltransferases. In some embodiments, the methyltransferase is MjDim1. In some embodiments, a methyltransferase enzyme used to modify a methylated adenosine preferentially attaches the functional group to a methylated nitrogen atom relative to an unmethylated nitrogen atom.

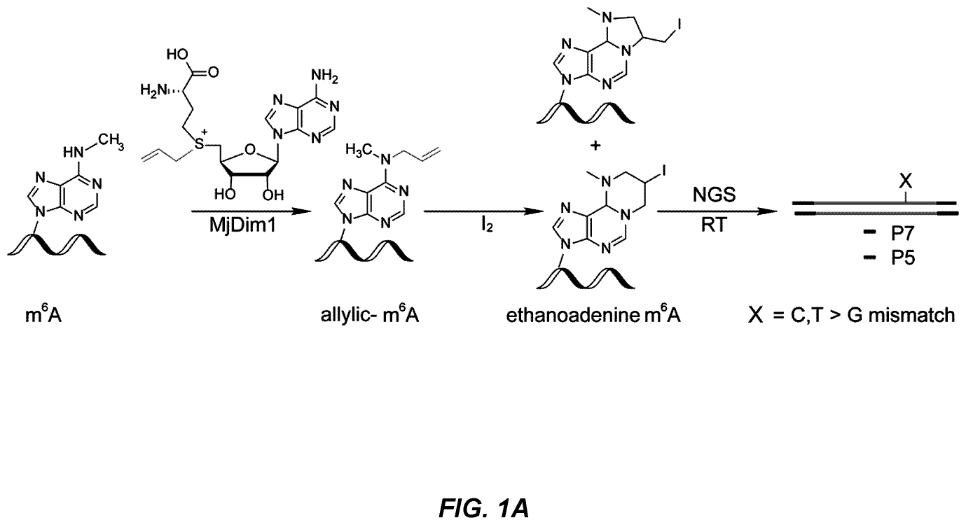

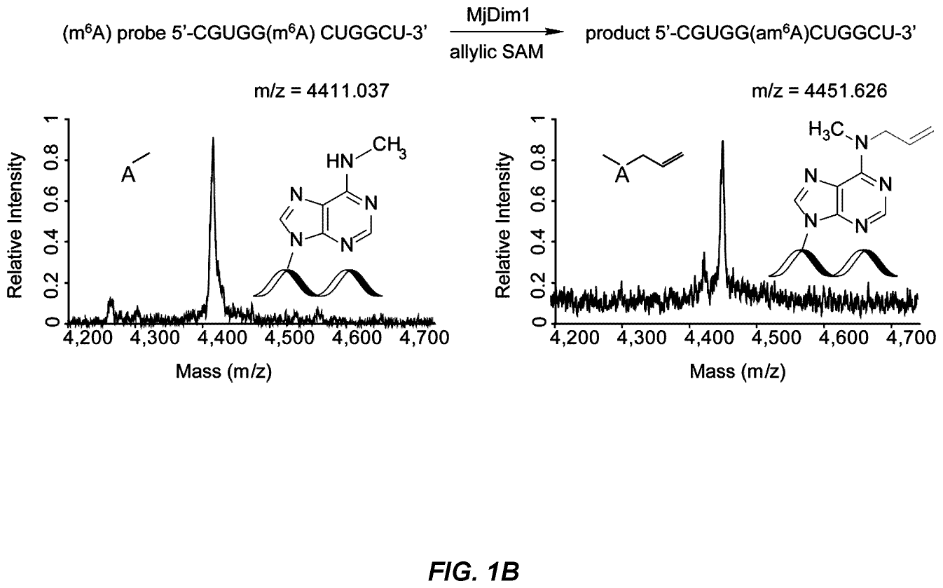

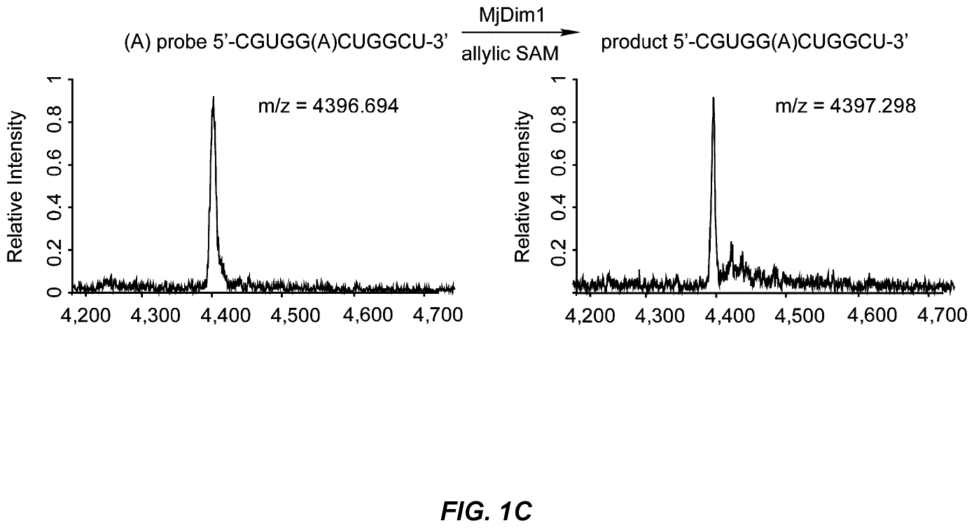

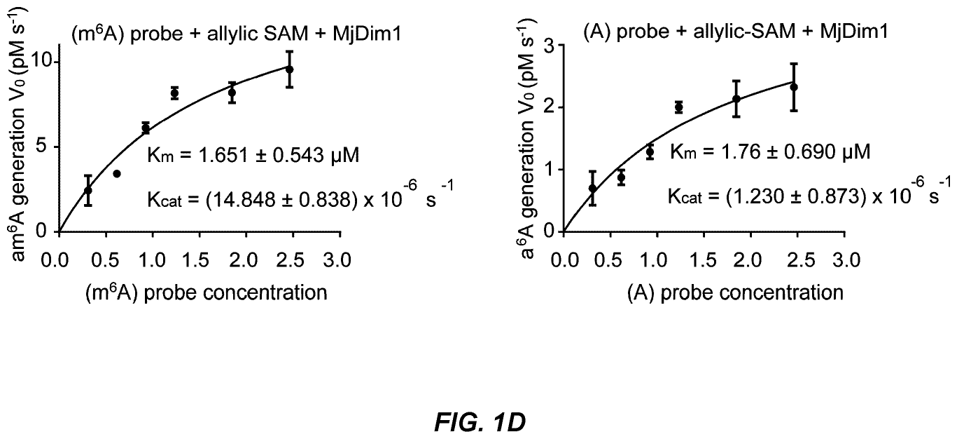

Modification of an N⁶-methyladenosine may further comprise incubating the modified N⁶-methyladenosine with a diatomic halogen molecule after attaching the functional group. In some embodiments, the diatomic halogen molecule is chlorine (Cl₂), bromine (Br₂), or iodine (I₂). In some embodiments, the modified N⁶-methyladenosine is incubated with I₂, thereby further modifying the functional group. For example, in cases where the functional group comprises an alkene, incubation with the I₂ may cyclize the functional group and/or attach the iodine to the N⁶-methyladenosine. FIG. 1A shows example reactions of the present disclosure for modifying an N⁶-methyladenosine.

B. Methyltransferase Enzymes

Embodiments of the present disclosure comprise methyltransferase enzymes. Methyltransferase enzymes may be useful in methods of the present disclosure, including methods for modifying a nitrogenous base, methods for modifying a methylated adenosine, and methods for detecting a methylated nucleotide. In some embodiments, a methyltransferase enzyme describes an enzyme belonging to the Enzyme Commission (EC) classification EC 2.1.1. In some embodiments, a methyltransferase enzyme describes an enzyme capable of facilitating transfer of a methyl group or other functional group from S-adenosylmethionine (SAM), or a derivative or analog thereof, to a nitrogenous base, nucleoside, and/or nucleotide. Methyltransferase enzymes may be natural or engineered. A methyltransferase enzyme may be a DNA methyltransferase or an RNA methyltransferase. A methyltransferase may be a dimethyltransferase, capable of transferring two methyl groups or functional groups. Example dimethyltransferase enzymes include Dim1/KsgA dimethyltransferase enzymes, such as Dim1 (EC 2.1.1.183, e.g., HsDim1, ScDim1, or MjDim1) or KsgA (EC 2.1.1.182).

Methyltransferase enzymes useful in the present methods (e.g., methods for methylated nucleotide detection) include those with preference for methylated nitrogenous bases over unmethylated nitrogenous bases. Such preference may be determined based on the functional group (e.g., methyl, allyl, etc.) used in the reaction. For example, as disclosed herein, the dimethyltransferase MjDim1 shows preference for methylated N⁶-methyladenosine compared with unmethylated adenosine when transferring an allyl group from a SAM analog. Thus, methods of the present disclosure include subjecting nucleic acids comprising N⁶-methyladenosine (e.g., mRNA) to conditions sufficient to preferentially attach a functional group, such as an allyl group, to N⁶-methyladenosine versus unmethylated adenosine.

C. S-adenosyl-1-Methionine and Analogs

Aspects of the present disclosure relate to S-adenosyl-1-methionine (SAM) and analogs thereof. In some embodiments, disclosed herein are SAM analogs comprising one or more functional groups. A SAM analog may comprise a functional group which is not a methyl group in place of the methyl group found in natural SAM. A functional group describes any chemical moiety which may be attached to a SAM molecule to generate an analog. Functional groups which may be used in the disclosed methods and compositions include chemical moieties having at least two carbon atoms. Example functional groups include alkyl groups and olefinic groups having at least two carbons. In some embodiments, a functional group is not a methyl group. In some embodiments, a functional group comprises an alkene. In some embodiments, a functional group is an allyl group. SAM analogs may be useful in attachment of a functional group to a nitrogenous base using a methyltransferase enzyme. In one embodiment, the SAM analog has the formula:

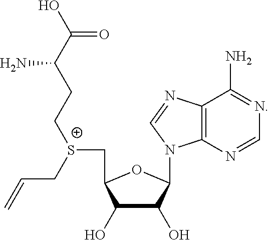

SAM analogs may be used in methyltransferase reactions of the present disclosure. For example, a SAM analog comprising a functional group may be provided together with a methyltransferase enzyme under conditions sufficient to attach the functional group to a methylated nitrogenous base (e.g., N⁶-methyladenosine). A SAM analog of the present disclosure may be provided as a part of compositions or kits useful in detection of RNA methylation.

### II. Sample Preparation

In certain aspects, methods involve obtaining a sample from a subject. The methods of obtaining provided herein may include methods of biopsy such as fine needle aspiration, core needle biopsy, vacuum assisted biopsy, incisional biopsy, excisional biopsy, punch biopsy, shave biopsy, liquid biopsy, or skin biopsy. In certain embodiments the sample is obtained from a biopsy from esophageal tissue by any of the biopsy methods previously mentioned. In other embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, sweat, hair follicle, buccal tissue, tears, menses, feces, or saliva. In certain aspects of the current methods, any medical professional such as a doctor, nurse or medical technician may obtain a biological sample for testing. Yet further, the biological sample can be obtained without the assistance of a medical professional.

A biological sample may include but is not limited to, tissue, cells, or biological material from cells or derived from cells of a subject. In some embodiments, a biological sample comprises extracellular vesicles such as exosomes. The biological sample may be a heterogeneous or homogeneous population of cells or tissues. A biological sample may be a cell-free sample. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, cerebrospinal fluid collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as multiple esophageal samples may be obtained for diagnosis by the methods described herein. In other cases, multiple samples, such as one or more samples from one tissue type (for example esophagus) and one or more samples from another specimen (for example serum) may be obtained for diagnosis by the methods. In some cases, multiple samples such as one or more samples from one tissue type (e.g. esophagus) and one or more samples from another specimen (e.g. serum) may be obtained at the same or different times. Samples may be obtained at different times are stored and/or analyzed by different methods. For example, a sample may be obtained and analyzed by routine staining methods or any other cytological analysis methods.

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

### III. Assay Methods

A. Detection of Methylated RNA

Aspects of the methods include assaying nucleic acids to determine expression levels and/or methylation levels of nucleic acids. In some embodiments, methods of the present disclosure comprise detection of RNA methylation. Embodiments of the disclosure include the detection of one or more methylated nucleotides, such as at least, at most, or exactly 1, 2, 3, 4, 5, 6, 7, 8, 9, or 10 methylated nucleotides (or any range derivable therein) per RNA molecule. Methylated nucleotides that may be detected using methods of the present disclosure include methylated adenosine (e.g., N⁶-methyladenosine). In some embodiments, disclosed herein are methods of detecting N⁶-methyladenosine (m⁶A) in RNA from a biological sample.

In some embodiments, a method for detecting m⁶A in an RNA molecule comprises incubating the RNA molecule with a methyltransferase enzyme and a S-adenosyl-1-methionine (SAM) analog comprising a functional group under conditions sufficient to attach the functional group to the m⁶A, thereby generating a modified m⁶A. Sufficient conditions for attachment of a functional group include sufficient buffer conditions, salt conditions, temperature conditions, etc., which allow the methyltransferase enzyme to transfer the functional group from the SAM analog to the m⁶A. Conditions sufficient for enzymatic reactions, including methyltransferase reactions, are known or may be readily experimentally determined by one skilled in the art. In one embodiment, an allyl group is attached to a m⁶A, thereby generating allylic-m⁶A.

In some embodiments, a m⁶A may be further modified by treatment with a diatomic halogen molecule. A diatomic halogen molecule may be, for example, chlorine (Cl₂), bromine (Br₂), or iodine (I₂). In some embodiments, the diatomic halogen molecule is iodine (I₂). In some embodiments, treatment with a diatomic halogen molecule serves to attach a halogen atom from the diatomic halogen molecule to a functional group comprising an alkene. In some embodiments, methods comprise incubation of a allyl-m⁶A with I₂ to generate ethanoadenine m⁶A (see FIG. 1A).

Following attachment of the functional group and, in some cases, additional reactions (e.g., treatment with diatomic halogen molecules), the RNA molecule may be subjected to conditions sufficient to generate a complementary nucleic acid molecule. Conditions include, for example, reverse transcription conditions, which may comprise providing a reverse transcriptase enzyme and conditions sufficient to perform reverse transcription on the RNA molecule to generate a complementary DNA (cDNA) molecule. A reverse transcriptase (RT) may describe an enzyme having EC classification EC 2.7.7.49. In some embodiments, the RT is an HIV RT or variant thereof, an M-MuLV RT or variant thereof, an AMV RT or variant thereof, a Bst polymerase (e.g., Bst, Bst 2.0, or Bst 3.0) or variant thereof, or a Klentaq polymerase or variant thereof. In some embodiments, the RT is an HIV RT. In some embodiments, the RT is a Bst polymerase or functional fragment thereof. In some embodiments, the RT is Bst 2.0 DNA polymerase. In some embodiments, the polymerase is a Klentaq polymerase or functional fragment thereof. In some embodiments, the RT is an RT having a preference for methylated adenosine over unmethylated adenosine. A cDNA molecule obtained from reverse transcription of an RNA molecule comprising a m⁶A may comprise a mutation at a residue in the cDNA molecule corresponding to the m⁶A from the RNA molecule.

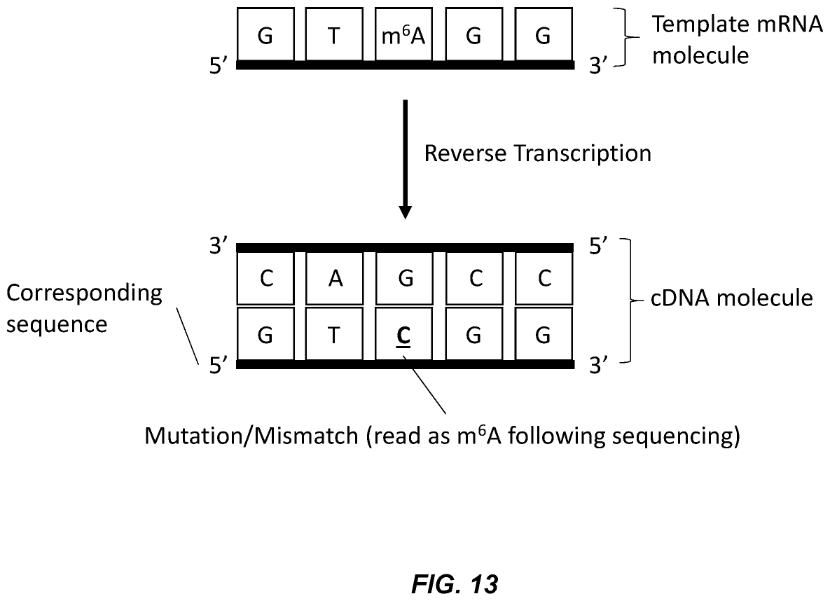

A mutation at a residue in a nucleic acid molecule (e.g., cDNA molecule) derived from a template nucleic acid molecule describes a nucleotide which is not identical to the nucleotide at the corresponding residue in the template nucleic acid molecule. For example, where a template mRNA molecule has an “A” nucleotide (or variant thereof) at a residue, a mutation in a cDNA molecule generated from the mRNA molecule describes the presence of a nucleotide other than an “A” (or variant thereof) at the corresponding residue (e.g., the presence of a “G”, “T”, or “C” nucleotide). In one example, as depicted in FIG. 13, a template mRNA molecule has the sequence 5′-GTm⁶AGG-3′ and a cDNA molecule generated from the mRNA molecule has a corresponding sequence 5′-GTCGG-3′. In this example, the cDNA molecule has a mutation at the third position (i.e., at the “C” nucleotide). The mutation corresponds to and can be used to identify the presence and location of the m⁶A in the template mRNA sequence. For example, by comparing the cDNA sequence to a reference database comprising the sequence of the mRNA molecule, the “C” nucleotide at the third position can be identified as a mutation based on the difference from the position in the reference database (i.e., based on the presence of the “C” nucleotide in the cDNA sequence instead of an “A” nucleotide as in the reference database), thereby identifying the template mRNA molecule as comprising an m⁶A at the third position.

The presence of a functional group on an m⁶A of an RNA molecule may induce generation of a mutation on a cDNA molecule. In some embodiments, modification of a functional group attached to an m⁶A by treatment with diatomic halogen molecules is required to generate a modified m⁶A having sufficient size to induce generation of mutations in a corresponding cDNA molecule obtained by reverse transcription of the RNA molecule. In some embodiments, modification of a functional group by treatment with a diatomic halogen molecule is not required to induce generation of a mutation.

Once generated, a nucleic acid molecule (e.g., cDNA molecule) may be sequenced. Example sequencing methods are described elsewhere herein. Sequencing may comprise amplification of the complementary nucleic acid molecule (e.g., via PCR or other amplification method). In some embodiments, sequencing generates a sequence corresponding to the nucleic acid molecule. A sequence may comprise the mutation corresponding to the methylated nucleotide in the RNA molecule. A sequence comprising a mutation may be compared to a template or control sequence derived from an unmodified RNA molecule. An unmodified RNA molecule may be from the same sample or a different sample as the modified RNA molecule. The sequence may be compared to the control sequence to identity the mutation and correlate the mutation with the m⁶A.

In embodiments comprising analysis of mRNA molecules comprising an m⁶A, prior to attaching a functional group to an m⁶A, an oligo-dT primer may be provided to the mRNA molecule under conditions sufficient to anneal the primer to the mRNA, thereby generating a double stranded region. An oligo-dT primer describes an oligonucleotide primer comprising a poly-deoxythimine region which is capable of hybridizing to a poly-adenylated region of an mRNA. An oligo-dT primer may be single stranded. Following generation of the double stranded region, a nuclease may be provided under conditions sufficient to digest the double stranded region. The nuclease may be a DNA nuclease. The nuclease may be a nuclease capable of specifically digesting a region of RNA when hybridized to DNA. The nuclease may be RNase H.

In some embodiments, RNA is obtained from a sample, such as a biological sample from a subject. In some embodiments, a portion of the RNA is subjected to sequencing in the absence of any treatment of modification. The portion may serve as a template or control for comparison with RNA modified via the disclosed methods. Such a template or control can enable the removal of “false positive” results resulting from modification of unmethylated nucleotides.

Examples of methylated RNA which may be modified and/or analyzed using compositions and methods of the present disclosure include messenger RNA (mRNA), transfer RNA (tRNA), ribosomal RNA (rRNA), long noncoding RNA (lncRNA), short noncoding RNA (sncRNA), microRNA (miRNA), small nuclear RNA (snRNA), small nucleolar RNA (snoRNA), small interfering RNA (siRNA), and short hairpin RNA (shRNA). RNA may be cell-free RNA (cfRNA).

In some embodiments, methylated RNA may be modified and analyzed in vitro. In some embodiments, methylated RNA may be modified and analyzed in situ (e.g., within a tissue sample). For example, methylated RNA comprising a methylated adenosine (e.g., m⁶A) may be modified and subjected to reverse transcription in situ, thereby generating a cDNA molecule comprising a mutation at a residue corresponding to the methylated adenosine. cDNA may then be detected using nucleic acid probes (e.g., fluorescent in situ hybridization (FISH) probes) designed to bind to the cDNA comprising the mutation but not to cDNA which does not comprise the mutation, thereby identifying the location of methylated RNA in a tissue sample.

B. Detection of Methylated DNA

Aspects of the methods include assaying nucleic acids to determine expression levels and/or methylation levels of nucleic acids. In some embodiments, methods of the present disclosure comprise detection of DNA methylation. In some embodiments, detection of DNA methylation comprises detection of methylated cytosine (e.g., 5-mC). In some embodiments, detection of DNA methylation comprises detection of methylated adenosine (e.g., m⁶A). Certain assays for the detection of methylated DNA are known in the art. Exemplary methods are described herein. One or more of the described methods for detection of methylated DNA may be used, alone or in combination.

1. HPLC-UV

The technique of HPLC-UV (high performance liquid chromatography-ultraviolet), developed by Kuo and colleagues in 1980 (described further in Kuo K. C. et al., Nucleic Acids Res. 1980; 8:4763-4776, which is herein incorporated by reference) can be used to quantify the amount of deoxycytidine (dC) and methylated cytosines (5 mC) present in a hydrolysed DNA sample. The method includes hydrolyzing the DNA into its constituent nucleoside bases, the 5 mC and dC bases are separated chromatographically and, then, the fractions are measured. Then, the 5 mC/dC ratio can be calculated for each sample, and this can be compared between the experimental and control samples.

2. LC-MS/MS

Liquid chromatography coupled with tandem mass spectrometry (LC-MS/MS) is an high-sensitivity approach to HPLC-UV, which requires much smaller quantities of the hydrolysed DNA sample. In the case of mammalian DNA, of which ˜2%-5% of all cytosine residues are methylated, LC-MS/MS has been validated for detecting levels of methylation levels ranging from 0.05%-10%, and it can confidently detect differences between samples as small as ˜0.25% of the total cytosine residues, which corresponds to ˜5% differences in global DNA methylation. The procedure routinely requires 50-100 ng of DNA sample, although much smaller amounts (as low as 5 ng) have been successfully profiled. Another major benefit of this method is that it is not adversely affected by poor-quality DNA (e.g., DNA derived from FFPE samples).

3. ELISA-Based Methods

There are several commercially available kits, all enzyme-linked immunosorbent assay (ELISA) based, that enable the quick assessment of DNA methylation status. These assays include Global DNA Methylation ELISA, available from Cell Biolabs; Imprint Methylated DNA Quantification kit (sandwich ELISA), available from Sigma-Aldrich; EpiSeeker methylated DNA Quantification Kit, available from abcam; Global DNA Methylation Assay—LINE-1, available from Active Motif; 5-mC DNA ELISA Kit, available from Zymo Research; MethylFlash Methylated DNAS-mC Quantification Kit and MethylFlash Methylated DNAS-mC Quantification Kit, available from Epigentek. These kits may be used using instructions provided by the manufacturer, or may be modified or combined at necessary depending on the application. For example, a kit designed for detection of 5-mC using an anti-5mC antibody may be modified by replacing the anti-5mC antibody with an anti-m⁶A antibody for the detection of methylated adenosine.

Briefly, the DNA sample is captured on an ELISA plate, and the methylated nucleotides (e.g., cytosine and/or adenosine) are detected through sequential incubations steps with: (1) a primary antibody raised against a methylated nucleotide (e.g., 5-mC, m⁶A); (2) a labelled secondary antibody; and then (3) colorimetric/fluorometric detection reagents.

The Global DNA Methylation Assay—LINE-1 specifically determines the methylation levels of LINE-1 (long interspersed nuclear elements-1) retrotransposons, of which ˜17% of the human genome is composed. These are well established as a surrogate for global DNA methylation. Briefly, fragmented DNA is hybridized to biotinylated LINE-1 probes, which are then subsequently immobilized to a streptavidin-coated plate. Following washing and blocking steps, methylated nucleotides (e.g., cytosine and/or adenosine) are quantified using an antibody specific for one or more methylated nucleotides of interest (e.g., anti-5 mC antibody, anti-m⁶A antibody, etc.), HRP-conjugated secondary antibody and chemiluminescent detection reagents. Samples are quantified against a standard curve generated from standards with known LINE-1 methylation levels. The manufacturers claim the assay can detect DNA methylation levels as low as 0.5%. Thus, by analyzing a fraction of the genome, it is possible to achieve better accuracy in quantification.

4. LINE-1 Pyrosequencing

Levels of LINE-1 methylation can alternatively be assessed by another method that involves the bisulfite conversion of DNA, followed by the PCR amplification of LINE-1 conservative sequences for the detection of methylated cytosine. The methylation status of the amplified fragments is then quantified by pyrosequencing, which is able to resolve differences between DNA samples as small as ˜5%. The method is particularly well suited for high throughput analysis of cancer samples, where hypomethylation is very often associated with poor prognosis. This method is particularly suitable for human DNA, but there are also versions adapted to rat and mouse genomes.

5. AFLP and RFLP

Detection of fragments that are differentially methylated could be achieved by traditional PCR-based amplification fragment length polymorphism (AFLP), restriction fragment length polymorphism (RFLP) or protocols that employ a combination of both.

6. Bisulfite Sequencing

In some embodiments, methods comprise the use of bisulfite sequencing for the detection of methylated cytosines (e.g., 5-mC). The bisulfite treatment of DNA mediates the deamination of cytosine into uracil, and these converted residues will be read as thymine, as determined by PCR-amplification and subsequent sequencing analysis. However, 5 mC residues are resistant to this conversion and, so, will remain read as cytosine. Thus, comparing the Sanger sequencing read from an untreated DNA sample to the same sample following bisulfite treatment enables the detection of the methylated cytosines. With the advent of next-generation sequencing (NGS) technology, this approach can be extended to DNA methylation analysis across an entire genome. To ensure complete conversion of non-methylated cytosines, controls may be incorporated for bisulfite reactions.

Whole genome bisulfite sequencing (WGBS) is similar to whole genome sequencing, except for the additional step of bisulfite conversion. Sequencing of the 5 mC-enriched fraction of the genome is not only a less expensive approach, but it also allows one to increase the sequencing coverage and, therefore, precision in revealing differentially-methylated regions. Sequencing could be done using any existing NGS platform; Illumina and Life Technologies both offer kits for such analysis.

Bisulfite sequencing methods include reduced representation bisulfite sequencing (RRBS), where only a fraction of the genome is sequenced. In RRBS, enrichment of CpG-rich regions is achieved by isolation of short fragments after MspI digestion that recognizes CCGG sites (and it cut both methylated and unmethylated sites). It ensures isolation of ˜85% of CpG islands in the human genome. Then, the same bisulfite conversion and library preparation is performed as for WGBS. The RRBS procedure normally requires ˜100 ng-1 μg of DNA.

7. Methods that Exclude Bisulfite Conversion

In some aspects, direct detection of modified bases without bisulfite conversion may be used to detect methylation. Pacific Biosciences has developed a way to detect methylated bases directly by monitoring the kinetics of polymerase during single molecule sequencing and offers a commercial product for such sequencing (further described in Flusberg B. A., et al., Nat. Methods. 2010; 7:461-465, which is herein incorporated by reference). Other methods include single-molecule real-time sequencing technology (SMRT) and Nanopore sequencing, each of which is able to detect modified bases directly (described in, for example, Laszlo A. H. et al., Proc. Natl. Acad. Sci. USA. 2013, Schreiber J., et al., Proc. Natl. Acad. Sci. USA. 2013, and Peng N. et al., Bioinformatics. 2019, which are herein incorporated by reference). In some embodiments, nanopore sequencing is used to directly sequence RNA containing methylated adenosine, without the need for reverse transcription.

8. Array or Bead Hybridization

Methylated DNA fractions of the genome, usually obtained by immunoprecipitation, could be used for hybridization with microarrays. Currently available examples of such arrays include: the Human CpG Island Microarray Kit (Agilent), the GeneChip Human Promoter 1.0R Array and the GeneChip Human Tiling 2.0R Array Set (Affymetrix).

The search for differentially-methylated regions using bisulfite-converted DNA could be done with the use of different techniques. Some of them are easier to perform and analyse than others, because only a fraction of the genome is used. The most pronounced functional effect of DNA methylation occurs within gene promoter regions, enhancer regulatory elements and 3′ untranslated regions (3′UTRs). Assays that focus on these specific regions, such as the Infinium HumanMethylation450 Bead Chip array by Illumina, can be used. The arrays can be used to detect methylation status of genes, including miRNA promoters, 5′ UTR, 3′ UTR, coding regions (˜17 CpG per gene) and island shores (regions ˜2 kb upstream of the CpG islands).

Briefly, bisulfite-treated genomic DNA is mixed with assay oligos, one of which is complimentary to uracil (converted from original unmethylated cytosine), and another is complimentary to the cytosine of the methylated (and therefore protected from conversion) site. Following hybridization, primers are extended and ligated to locus-specific oligos to create a template for universal PCR. Finally, labelled PCR primers are used to create detectable products that are immobilized to bar-coded beads, and the signal is measured. The ratio between two types of beads for each locus (individual CpG) is an indicator of its methylation level.

It is possible to purchase kits that utilize the extension of methylation-specific primers for validation studies. In the VeraCode Methylation assay from Illumina, 96 or 384 user-specified CpG loci are analysed with the GoldenGate Assay for Methylation. Differently from the BeadChip assay, the VeraCode assay requires the BeadXpress Reader for scanning.

9. Methyl-Sensitive Cut Counting: Endonuclease Digestion Followed by Sequencing

As an alternative to sequencing a substantial amount of methylated (or unmethylated) DNA, one could generate snippets from these regions and map them back to the genome after sequencing. Moreover, coverage in NGS could be good enough to quantify the methylation level for particular loci. The technique of serial analysis of gene expression (SAGE) has been adapted for this purpose and is known as methylation-specific digital karyotyping, as well as a similar technique, called methyl-sensitive cut counting (MSCC).

In summary, in all of these methods, methylation-sensitive endonuclease(s), e.g., HpaII is used for initial digestion of genomic DNA in unmethylated sites followed by adaptor ligation that contains the site for another digestion enzyme that is cut outside of its recognized site, e.g., EcoP15I or MmeI. These ways, small fragments are generated that are located in close proximity to the original HpaII site. Then, NGS and mapping to the genome are performed. The number of reads for each HpaII site correlates with its methylation level.

Recently, a number of restriction enzymes have been discovered that use methylated DNA as a substrate (methylation-dependent endonucleases). Most of them were discovered and are sold by SibEnzyme: BisI, BlsI, GlaI. GluI, KroI, MteI, PcsI, PkrI. The unique ability of these enzymes to cut only methylated sites has been utilized in the method that achieved selective amplification of methylated DNA. Three methylation-dependent endonucleases that are available from New England Biolabs (FspEI, MspJI and LpnPI) are type IIS enzymes that cut outside of the recognition site and, therefore, are able to generate snippets of 32 bp around the fully-methylated recognition site that contains CpG. These short fragments could be sequences and aligned to the reference genome. The number of reads obtained for each specific 32-bp fragment could be an indicator of its methylation level. Similarly, short fragments could be generated from methylated CpG islands with Escherichia coli's methyl-specific endonuclease McrBC, which cuts DNA between two half-sites of (G/A) mC that are lying within 50 bp-3000 bp from each other. This is a very useful tool for isolation of methylated CpG islands that again can be combined with NGS. Being bisulfite-free, these three approaches have a great potential for quick whole genome methylome profiling.

C. Sequencing

Aspects of the present disclosure include nucleic acid sequencing. Nucleic acid sequencing may be used for detection and analysis of one or more nucleic acids in a sample. In some embodiments, the disclosed methods comprise sequencing nucleic acids from a sample to detect one or more genetic mutations or abnormalities (e.g., insertions, deletions, frameshift mutations, single nucleotide polymorphisms (SNPs), chromosomal abnormalities (e.g., inversions, substitutions, copy number variations.), etc.). In some embodiments, the disclosed methods comprise sequencing nucleic acids from a sample to detect methylation (e.g., DNA methylation, RNA methylation). Sequencing may comprise whole genome sequencing and/or targeted sequencing. In some embodiments, the methods of the disclosure include a sequencing method. Exemplary sequencing methods include those described below.

1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform.

3. 454 Pyrosequencing.

A parallelized version of pyrosequencing was developed by 454 Life Sciences. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

4. Illumina (Solexa) Sequencing.

Solexa developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massively parallel sequencing technology based on “DNA Clusters”, which involves the clonal amplification of DNA on a surface.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

5. SOLiD Sequencing.

Applied Biosystems' SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent Semiconductor Sequencing.

Ion Torrent Systems Inc. developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

7. DNA Nanoball Sequencing.

DNA nanoball sequencing is a type of high throughput sequencing technology used to determine the entire genomic sequence of an organism. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.

10. Nanopore Sequencing.

Nanopore sequencing is based on variations in ionic current generated as nucleic acid passes through a nanopore, such as a protein. Nucleic acid is passed through a nanopore in a membrane, and each change in current across the membrane is measured and correlated with a particular nucleotide. In some embodiments, nanopore sequencing is performed using sequencing systems developed by Oxford Nanopore Technologies. Nanopore sequencing is described in, for example, Wang Y. et al., Front. Genet., 2015, and Jain, M., et al., Genome Biol., 2016, each of which is incorporated herein by reference in its entirety. In some embodiments, nanopore sequencing is used to directly sequence RNA containing methylated adenosine, without the need for reverse transcription.

D. Additional Assay Methods

In some embodiments, methods involve amplifying and/or sequencing one or more target genomic regions using at least one pair of primers specific to the target genomic regions. In certain embodiments, the primers are heptamers. In other embodiments, enzymes are added such as primases or primase/polymerase combination enzyme to the amplification step to synthesize primers.

In some embodiments, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic acid amplification, polymerase chain reaction (PCR), quantitative PCR, RT-PCR, in situ hybridization, digital PCR, ddPCR (digital droplet PCR, also droplet digital PCR), nCounter (nanoString), BEAMing (Beads, Emulsions, Amplifications, and Magnetics) (Inostics), ARMS (Amplification Refractory Mutation Systems), RNA-Seq, TAm-Seg (Tagged-Amplicon deep sequencing), PAP (Pyrophosphorolysis-activation polymerization), next generation RNA sequencing, northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

Amplification primers or hybridization probes can be prepared to be complementary to a genomic region, biomarker, probe, or oligo described herein. The term “primer” or “probe” as used herein, is meant to encompass any nucleic acid that is capable of priming the synthesis of a nascent nucleic acid in a template-dependent process and/or pairing with a single strand of a polynucleotide of the disclosure, or portion thereof. Typically, primers are oligonucleotides from ten to twenty and/or thirty nucleic acids in length, but longer sequences can be employed. Primers may be provided in double-stranded and/or single-stranded form.

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one embodiment, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular nucleic acid or interest (e.g., nucleic acid biomarker).

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one embodiment, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one embodiment, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some embodiments, the density may be or may be at least 50, 100, 200, 300, 400, 500 or more probes/cm² (or any range derivable therein).

Specifically contemplated are chip-based nucleic acid technologies such as those described by Hacia et al. (1996) and Shoemaker et al. (1996). Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization (see also, Pease et al., 1994; and Fodor et al, 1991). It is contemplated that this technology may be used in conjunction with evaluating the expression level of one or more cancer biomarkers with respect to diagnostic, prognostic, and treatment methods.

Certain embodiments may involve the use of arrays or data generated from an array. Data may be readily available. Moreover, an array may be prepared in order to generate data that may then be used in correlation studies.

### IV. Methods of Use

A. Clinical and Diagnostic Applications

The methods of the disclosure may be useful for evaluating nucleic acid (e.g., DNA, RNA) for clinical and/or diagnostic purposes. Certain embodiments relate to a method for evaluating a sample comprising RNA molecules. Example RNA molecules which may be analyzed using the disclosed methods and compositions include messenger RNA (mRNA), transfer RNA (tRNA), ribosomal RNA (rRNA), long noncoding RNA (lncRNA), short noncoding RNA (sncRNA), microRNA (miRNA), small nuclear RNA (snRNA), small nucleolar RNA (snoRNA), small interfering RNA (siRNA), and short hairpin RNA (shRNA). The evaluation may be the detection or determination of a particular adenosine modification or the differential detection or determination of a particular modification.

In some embodiments, the methods of the disclosure can be used in the discovery of novel biomarkers for a disease or condition. In some embodiments, the methods of the disclosure can performed on a sample from a patient to provide a prognosis for a certain disease or condition in the patient. In some embodiments, the methods of the disclosure can be performed on a sample from a patient to predict the patient's response to a particular therapy. In some embodiments, the disease comprises a cancer. For example, the cancer may be pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye Cancer, intraocular melanoma eye Cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, melanoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and Wilms tumor (kidney cancer).

In some embodiments, the cancer comprises ovarian, prostate, colon, or lung cancer. In some embodiments, the method is for determining novel biomarkers for ovarian, prostate, colon, or lung cancer by evaluating cell-free nucleic acid (e.g., cell-free RNA) using methods of the disclosure. In some embodiments, the methods of the disclosure may be used on fetal RNA isolated from a pregnant female. In some embodiments, the methods of the disclosure may be used for prenantal diagnostics using fetal RNA isolated from a pregnant female. In some embodiments, the methods of the disclosure may be used for the evaluation of a fertilized embryo, such as a zygote or a blastocyst for the determination of embryo quality or for the presence or absence of a particular disease marker.

### V. Detecting a Genetic Signature

Particular embodiments concern the methods of detecting a genetic signature in an individual. In some embodiments, the method for detecting the genetic signature may include selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof, for example. The method for detecting the genetic signature may include fluorescent in situ hybridization, comparative genomic hybridization, arrays, polymerase chain reaction, sequencing, or a combination thereof, for example. The detection of the genetic signature may involve using a particular method to detect one feature of the genetic signature and additionally use the same method or a different method to detect a different feature of the genetic signature. Multiple different methods independently or in combination may be used to detect the same feature or a plurality of features.

A. Single Nucleotide Polymorphism (SNP) Detection

Particular embodiments of the disclosure concern methods of detecting a SNP in an individual. One may employ any of the known general methods for detecting SNPs for detecting the particular SNP in this disclosure, for example. Such methods include, but are not limited to, selective oligonucleotide probes, arrays, allele-specific hybridization, molecular beacons, restriction fragment length polymorphism analysis, enzymatic chain reaction, flap endonuclease analysis, primer extension, 5′-nuclease analysis, oligonucleotide ligation assay, single strand conformation polymorphism analysis, temperature gradient gel electrophoresis, denaturing high performance liquid chromatography, high-resolution melting, DNA mismatch binding protein analysis, surveyor nuclease assay, sequencing, or a combination thereof.

In some embodiments of the disclosure, the method used to detect the SNP comprises sequencing nucleic acid material from the individual and/or using selective oligonucleotide probes. Sequencing the nucleic acid material from the individual may involve obtaining the nucleic acid material from the individual in the form of genomic DNA, complementary DNA that is reverse transcribed from RNA, or RNA, for example. Any standard sequencing technique may be employed, including Sanger sequencing, chain extension sequencing, Maxam-Gilbert sequencing, shotgun sequencing, bridge PCR sequencing, high-throughput methods for sequencing, next generation sequencing, RNA sequencing, or a combination thereof. After sequencing the nucleic acid from the individual, one may utilize any data processing software or technique to determine which particular nucleotide is present in the individual at the particular SNP.

In some embodiments, the nucleotide at the particular SNP is detected by selective oligonucleotide probes. The probes may be used on nucleic acid material from the individual, including genomic DNA, complementary DNA that is reverse transcribed from RNA, or RNA, for example. Selective oligonucleotide probes preferentially bind to a complementary strand based on the particular nucleotide present at the SNP. For example, one selective oligonucleotide probe binds to a complementary strand that has an A nucleotide at the SNP on the coding strand but not a G nucleotide at the SNP on the coding strand, while a different selective oligonucleotide probe binds to a complementary strand that has a G nucleotide at the SNP on the coding strand but not an A nucleotide at the SNP on the coding strand. Similar methods could be used to design a probe that selectively binds to the coding strand that has a C or a T nucleotide, but not both, at the SNP. Thus, any method to determine binding of one selective oligonucleotide probe over another selective oligonucleotide probe could be used to determine the nucleotide present at the SNP.

One method for detecting SNPs using oligonucleotide probes comprises the steps of analyzing the quality and measuring quantity of the nucleic acid material by a spectrophotometer and/or a gel electrophoresis assay; processing the nucleic acid material into a reaction mixture with at least one selective oligonucleotide probe, PCR primers, and a mixture with components needed to perform a quantitative PCR (qPCR), which could comprise a polymerase, deoxynucleotides, and a suitable buffer for the reaction; and cycling the processed reaction mixture while monitoring the reaction. In one embodiment of the method, the polymerase used for the qPCR will encounter the selective oligonucleotide probe binding to the strand being amplified and, using endonuclease activity, degrade the selective oligonucleotide probe. The detection of the degraded probe determines if the probe was binding to the amplified strand.

Another method for determining binding of the selective oligonucleotide probe to a particular nucleotide comprises using the selective oligonucleotide probe as a PCR primer, wherein the selective oligonucleotide probe binds preferentially to a particular nucleotide at the SNP position. In some embodiments, the probe is generally designed so the 3′ end of the probe pairs with the SNP. Thus, if the probe has the correct complementary base to pair with the particular nucleotide at the SNP, the probe will be extended during the amplification step of the PCR. For example, if there is a T nucleotide at the 3′ position of the probe and there is an A nucleotide at the SNP position, the probe will bind to the SNP and be extended during the amplification step of the PCR. However, if the same probe is used (with a T at the 3′ end) and there is a G nucleotide at the SNP position, the probe will not fully bind and will not be extended during the amplification step of the PCR.

In some embodiments, the SNP position is not at the terminal end of the PCR primer, but rather located within the PCR primer. The PCR primer should be of sufficient length and homology in that the PCR primer can selectively bind to one variant, for example the SNP having an A nucleotide, but not bind to another variant, for example the SNP having a G nucleotide. The PCR primer may also be designed to selectively bind particularly to the SNP having a G nucleotide but not bind to a variant with an A, C, or T nucleotide. Similarly, PCR primers could be designed to bind to the SNP having a C or a T nucleotide, but not both, which then does not bind to a variant with a G, A, or T nucleotide or G, A, or C nucleotide respectively. In particular embodiments, the PCR primer is at least or no more than 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or more nucleotides in length with 100% homology to the template sequence, with the potential exception of non-homology the SNP location. After several rounds of amplifications, if the PCR primers generate the expected band size, the SNP can be determined to have the A nucleotide and not the G nucleotide.

B. Copy Number Variation Detection

Particular embodiments of the disclosure concern methods of detecting a copy number variation (CNV) of a particular allele. One can utilize any known method for detecting CNVs to detect the CNVs. Such methods include fluorescent in situ hybridization, comparative genomic hybridization, arrays, polymerase chain reaction, sequencing, or a combination thereof, for example. In some embodiments, the CNV is detected using an array, wherein the array is capable of detecting CNVs on the entire X chromosome and/or all targets of miR-362. Array platforms such as those from Agilent, Illumina, or Affymetrix may be used, or custom arrays could be designed. One example of how an array may be used includes methods that comprise one or more of the steps of isolating nucleic acid material in a suitable manner from an individual suspected of having the CNV and, at least in some cases from an individual or reference genome that does not have the CNV; processing the nucleic acid material by fragmentation, labelling the nucleic acid with, for example, fluorescent labels, and purifying the fragmented and labeled nucleic acid material; hybridizing the nucleic acid material to the array for a sufficient time, such as for at least 24 hours; washing the array after hybridization; scanning the array using an array scanner; and analyzing the array using suitable software. The software may be used to compare the nucleic acid material from the individual suspected of having the CNV to the nucleic acid material of an individual who is known not to have the CNV or a reference genome.

In some embodiments, detection of a CNV is achieved by polymerase chain reaction (PCR). PCR primers can be employed to amplify nucleic acid at or near the CNV wherein an individual with a CNV will result in measurable higher levels of PCR product when compared to a PCR product from a reference genome. The detection of PCR product amounts could be measured by quantitative PCR (qPCR) or could be measured by gel electrophoresis, as examples. Quantification using gel electrophoresis comprises subjecting the resulting PCR product, along with nucleic acid standards of known size, to an electrical current on an agarose gel and measuring the size and intensity of the resulting band. The size of the resulting band can be compared to the known standards to determine the size of the resulting band. In some embodiments, the amplification of the CNV will result in a band that has a larger size than a band that is amplified, using the same primers as were used to detect the CNV, from a reference genome or an individual that does not have the CNV being detected. The resulting band from the CNV amplification may be nearly double, double, or more than double the resulting band from the reference genome or the resulting band from an individual that does not have the CNV being detected. In some embodiments, the CNV can be detected using nucleic acid sequencing. Sequencing techniques that could be used include, but are not limited to, whole genome sequencing, whole exome sequencing, and/or targeted sequencing.

C. DNA Sequencing

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing using at least one bioinformatics tool, including VarScan2, any R package (including CopywriteR) and/or Annovar.

D. RNA Sequencing

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

E. Proteomics

In some embodiments, protein may be analyzed by mass spectrometry. The protein may be prepared for mass spectrometry using any method known in the art. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool.

### VI. Kits

The invention additionally provides kits for performing the methods of the disclosure. The contents of a kit can include one or more reagents described throughout the disclosure and/or one or more reagents known in the art for performing one or more steps described throughout the disclosure. For example, the kits may include one or more of the following: a S-adenosyl-1-methionine (SAM) analog, a methyltransferase (e.g., MjDim1), a reverse transcriptase (e.g., HIV reverse transcriptase, M-MuLV reverse transcriptase, Klentaq polymerase, Bst polymerase (e.g., Bst 2.0 polymerase, Bst 3.0 polymerase), etc.), a demethylase, a nuclease (e.g., RNase H), nuclease-free water, one or more primers, SPRI beads, magnetic beads, DNA polymerase, taq polymerase, dNTPs, DNA polymerase buffer, reverse transcriptase buffer, bivalent cations, monovalent cations, RNA polymerase, DTT, redox reagent, Mg²⁺, K⁺, Mn²⁺, adaptors, a protease, and/or NTPs.

The kits may include an agent or agents for modifying a methylated nitrogenous base, e.g., demethylase, SAM analog, etc.

One or more reagent is preferably supplied in a solid form or liquid buffer that is suitable for inventory storage, and later for addition into the reaction medium when the method of using the reagent is performed. Suitable packaging is provided. The kit may optionally provide additional components that are useful in the procedure. These optional components include buffers, capture reagents, developing reagents, labels, reacting surfaces, means for detection, control samples, instructions, and interpretive information.

Each kit may also include additional components that are useful for amplifying the nucleic acid, or sequencing the nucleic acid, or other applications of the present disclosure as described herein. The kit may optionally provide additional components that are useful in the procedure. These optional components include buffers, capture reagents, developing reagents, labels, reacting surfaces, means for detection, control samples, instructions, and interpretive information.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—m⁶A Labeling

Unlike N¹-methyladenosine (m¹A), which is located at the Watson-Crick face of the nucleobase and affects reverse transcription, N⁶-methyladenosine (m⁶A) loses all its modification information after reverse transcription. The chemical similarity between m⁶A and A makes it challenging to differentiate the two, and the inert characteristic of the methyl group on m⁶A precludes chemistry-based selective labeling. The Dim1/KsgA dimethyl transferase family can transfer four methyl groups from S-adenosyl-1-methionine (SAM) to two adenosines of the small subunit rRNA³. According to biochemistry studies, Methanocaldococcus jannaschii homolog Mjdim1 is the most efficient dimethyl-transferase among the three enzymes tested, and shows highly processive kinetics in converting m⁶A into m⁶₂A⁴. To develop methods and compositions for detecting m⁶A, the methyl group of SAM was replaced with an allyl group, thereby generating the analog allyl-SAM⁵.

Substitution of the SAM cofactor with allyl-SAM confers the Mjdim1 enzyme notable substrate preference for m⁶A over unmodified adenosine. FIG. 1B shows a matrix assisted laser desorption/ionization (MALDI) based mass spectrometry characterization of the shown m⁶A-containing 12mer template RNA treated with Mjdim1 and allyl-SAM. The extra molecular weight represents the allyl group. FIG. 1C shows a MALDI-based mass spectrometry characterization of the shown 12mer template RNA, which does not comprise any m⁶A. This data demonstrates a lack of extra molecular weight, showing that no new product was generated.

Allylic-modified m⁶A (am⁶A) can be chemically converted into N¹, N⁶-ethanoadenine (also ethanoadenine-m⁶A or EA) by I₂⁶. Following reverse transcription, a mutation may be generated at the residue corresponding to the EA. FIG. 1A shows a schematic representation of the conversion and mutation generation process. Following this process, m⁶A is chemically labeled and represented as mutations at the whole transcriptome level, while “non-specific” modification at unmodified adenosine sites remains low. FIG. 1D shows Michaelis-Menten steady-state kinetics of Mjdim1-catalyzed am⁶A and a⁶A modifications on Maldi_Probe_m⁶A and Maldi_Probe_A. The Kₘ is similar while the K_{cat} of the enzyme towards m⁶A is 10-fold that of unmodified adenosine.

### Example 2—Detection of N⁶-Methyladenosine with m⁶A-sac-seq

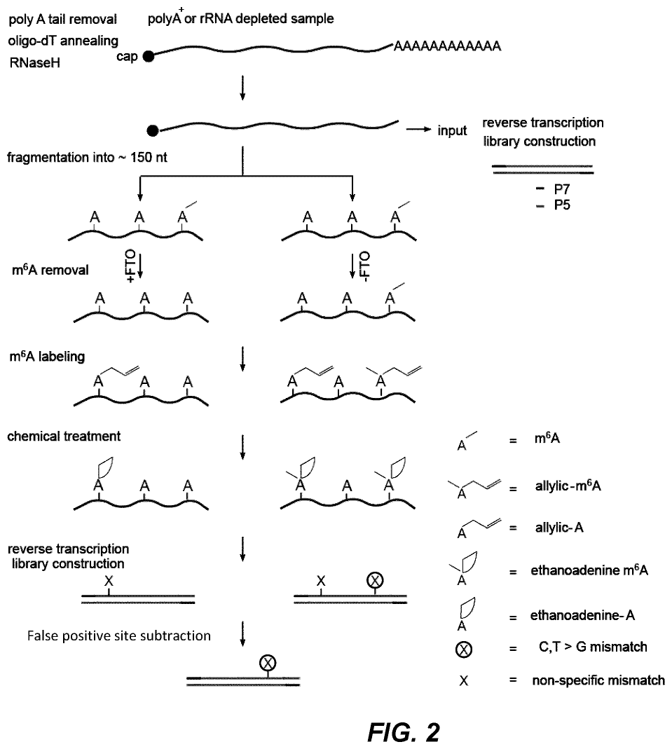

The general method described in Example 1 was named m⁶A selective allyl chemical labeling and sequencing, or m⁶A-sac-seq. An example procedure for an m⁶A-sac-seq process is shown in FIG. 2 and is as follows:
i) Purified RNA is annealed with oligo-dT, digested with RNase H to remove poly A tail followed by fragmentation into polynucleotide fragments with length of about 150 bp.
ii) A portion of RNA is subjected to library construction free of extra treatment as a reference input group.
iii) A portion of RNA is subjected to enzyme labeling with recombinant MjDim1 enzyme and allyl-SAM cofactor under optimized conditions as an experimental group.
iv) A portion of RNA sample is treated with FTO to erase m⁶A sites followed by enzyme labeling, considered as background noise group.
v) I₂ is added into recovered RNA to induce cyclization and formation of ethanoadenine-m⁶A homologue. cDNA is synthesized with reverse transcriptase (e.g., HIV reverse transcriptase) and the am⁶A site information is inferred from ethanoadenine-m⁶A-induced mutation. Comparison of results from treated RNA versus reference input and background noise group is used to accurately identify m⁶A sites in the transcriptome.

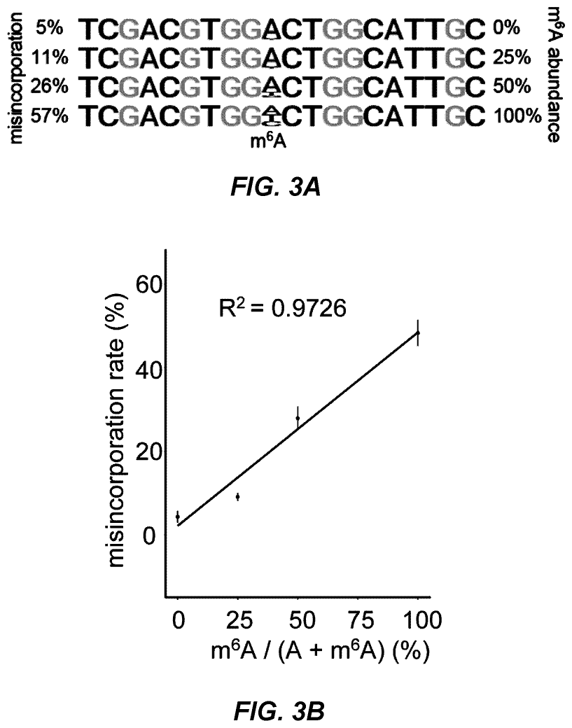

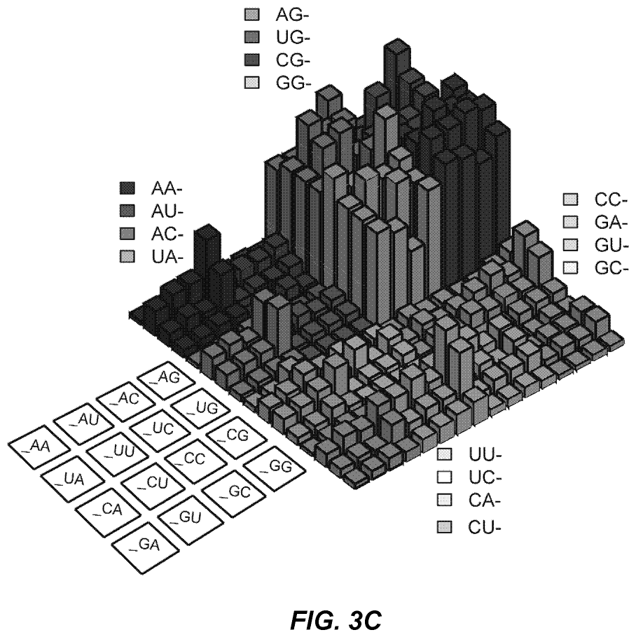

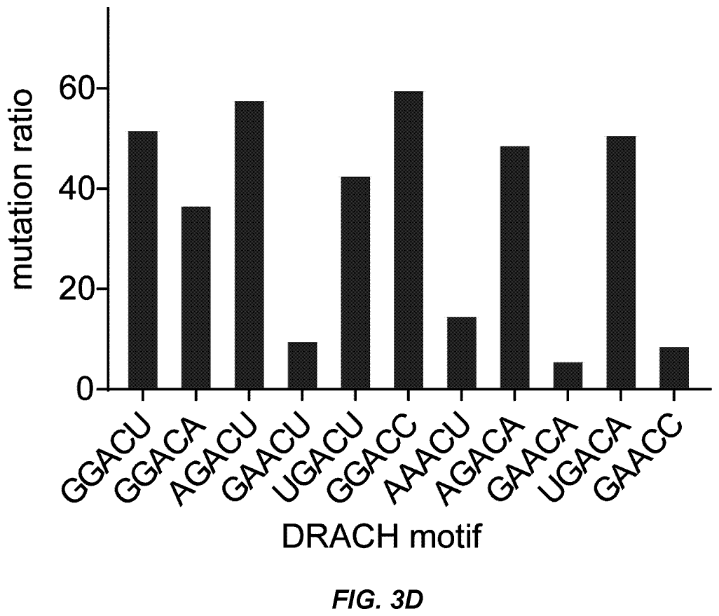

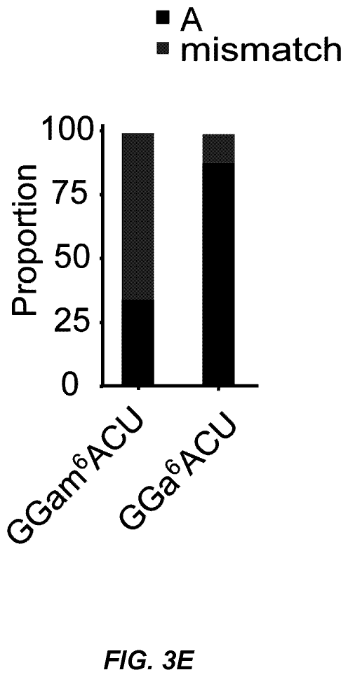

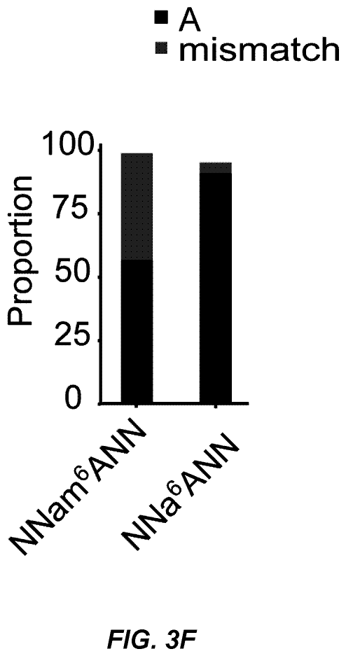

To identity the misincorporation pattern, m⁶A-sac-seq was performed with NNm⁶ANN-containing probes. FIG. 3C shows the sequence selectivity of Mjdim1. FIG. 3D shows the mutation ratio for the shown m⁶A consensus motifs (DRACH motif, where D=A, G, or U; R=purine; and H=A, C, or U), demonstrating that the method was able recover nearly all canonical m⁶A motifs. Motifs with Gm⁶AC tended to show higher mutation ratio (>30%) while mutation ratio of Am⁶AC containing motifs was relatively lower, though still significant (5%-10%).

Hela mRNA was mixed with a gradient of m⁶A-modified spike-in probes (0%, 25%, 50%, 100% 41 bp RNA probes) and subjected to m⁶A-sac-seq, followed by deep sequencing. The m⁶A sites in the spike-in probes indeed showed significant mutation rates compared with adjacent unmodified A/C/U/G sites (FIG. 3A). The mutation rate linearly correlated with m⁶A amount (R²=0.97) (FIG. 3B), demonstrating the quantitative capability of the method. Interestingly, am⁶A showed higher mutation rate compared with a⁶A. Thus, even in cases where an unmodified adenosine is allyl modified (i.e., non-specific modification), the resulting a⁶A results in about 10-fold less mutation compared with am⁶A (FIGS. 3E and 3F). FIG. 3E shows mismatch proportion using HIV RT enzyme induced by cyclized validation probes containing a GGam⁶ACU or GGa⁶ACU motif. FIG. 3F shows mismatch proportion using HIV RT enzyme induced by cyclized validation probes containing a NNam⁶ANN or NNa⁶ANN motif; NNam⁶ANN represents the specific m⁶A labeling product while NNa⁶ANN represents non-specific byproduct of A modification.

### Example 3—m⁶A Transcriptome Mapping and Validation

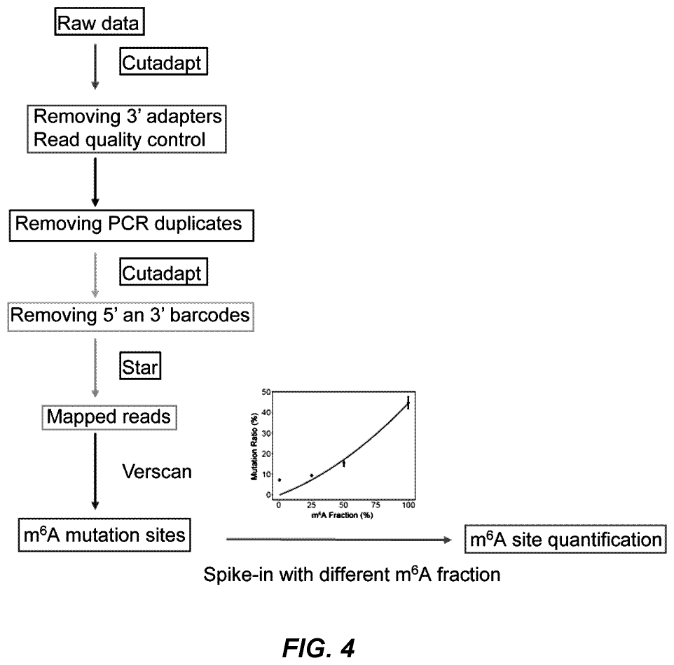

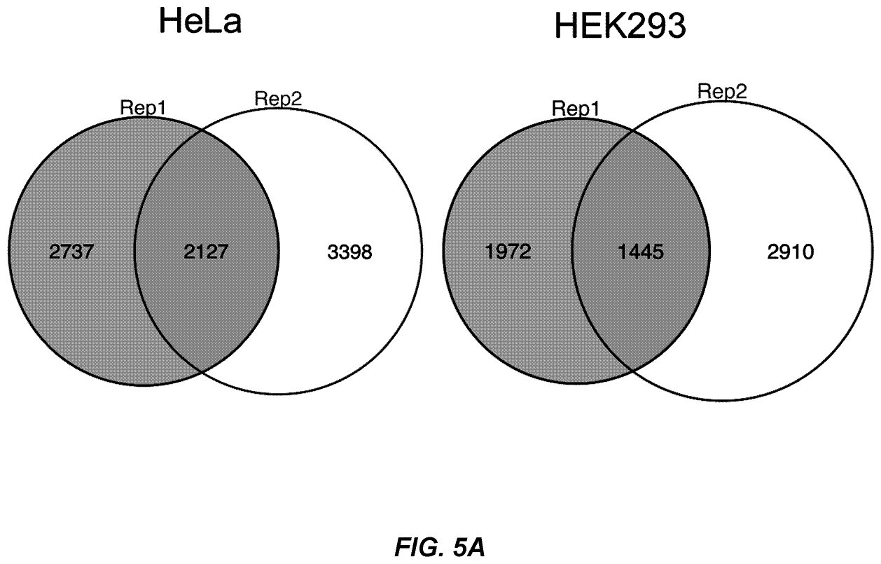

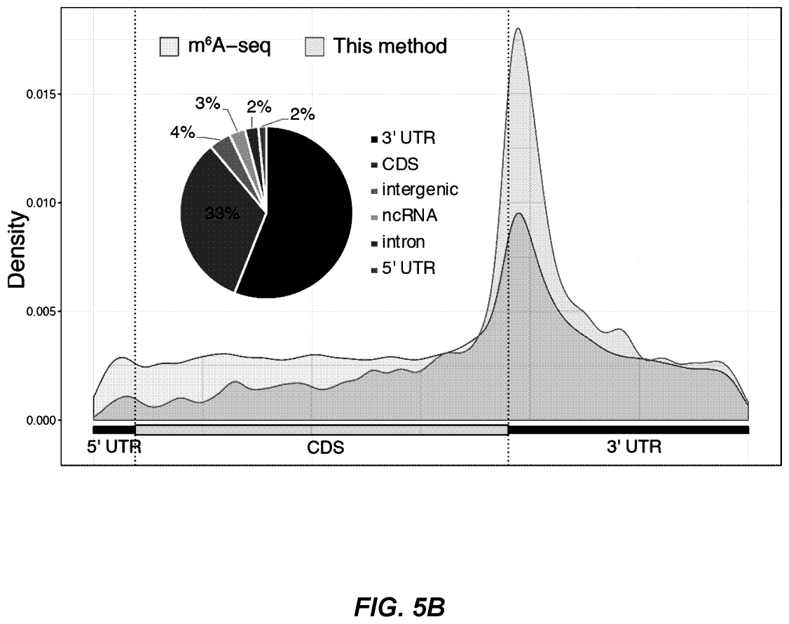

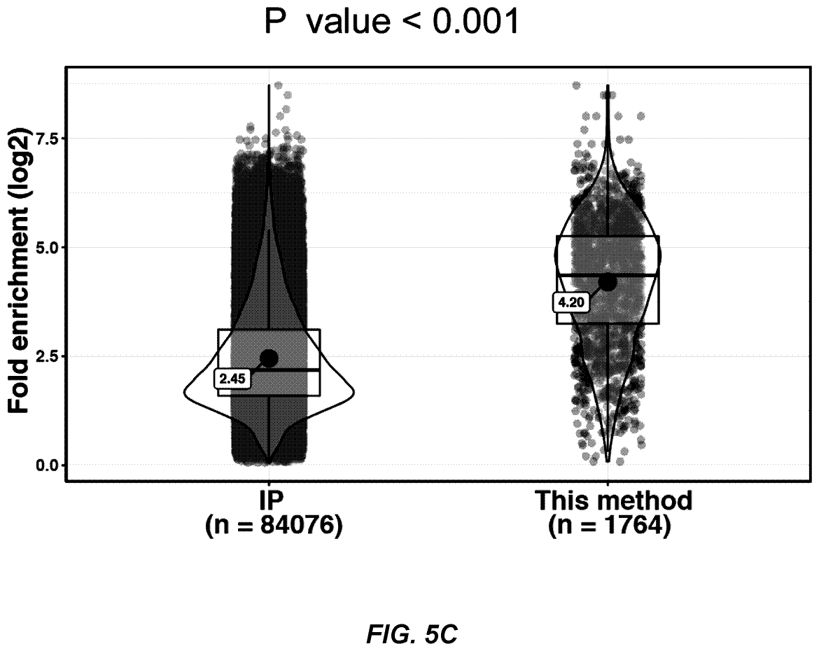

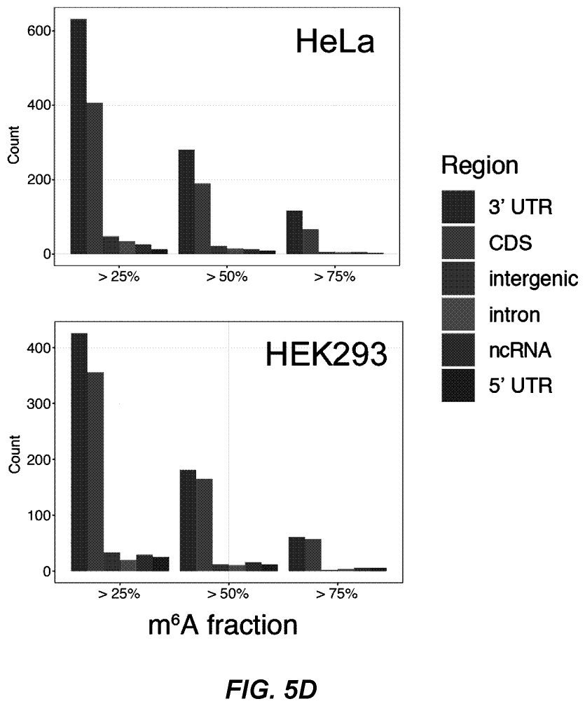

The m⁶A-sac-seq method was applied to map m⁶A sites in Hela and HEK cell transcriptomes. FIG. 4 shows a flowchart outlining the bioinformatics workflow process followed for m⁶A quantification. About 2000 highly confident and abundant m⁶A sites were identified. An overview of the identified sites is shown in FIG. 5A. FIG. 5B shows metagene profiles depicting sequence coverage in windows surrounding the stop codon; the pie chart represents the fraction of Hela m⁶A sites in each of six non-overlapping transcript segments. This data demonstrates that m⁶A sites are distributed canonically, enriched in the vicinity of the stop codon⁷. FIG. 5C shows that m⁶A sites are enriched in the high fold enrichment of MeRIP peaks. FIG. 5D shows the distribution of m⁶A in each of six non-overlapping transcript segments: 3′ UTR, CDS, intergenic, intron, ncRNA, and 5′UTR (shown left to right in each graph section).

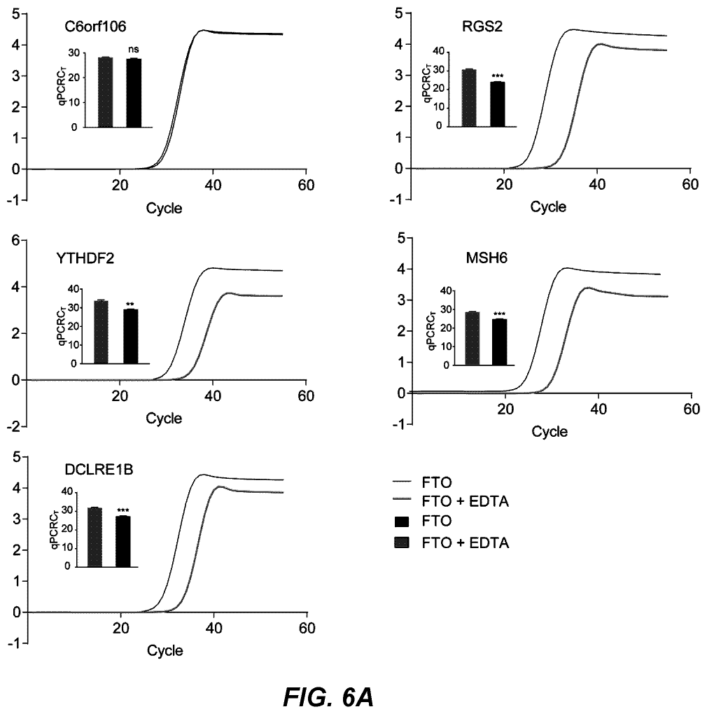

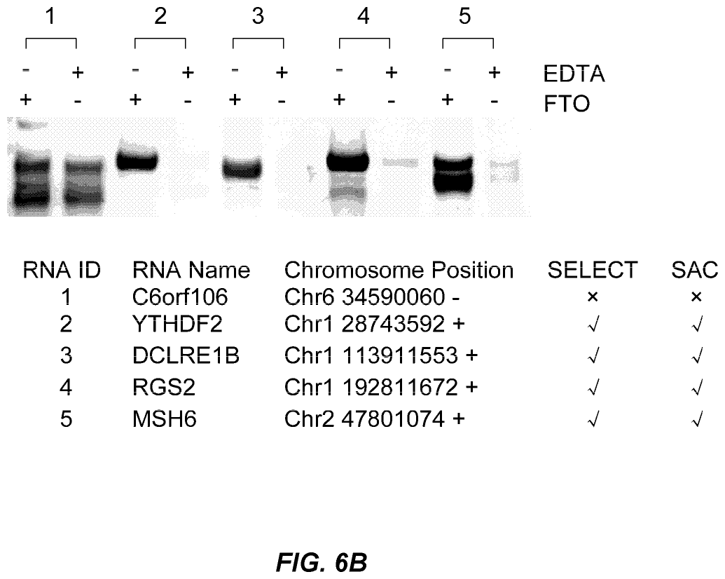

One m⁶A negative site and four m⁶A positive sites were chosen and validated using a SELECT method⁸. The results obtained with both methods were consistent (FIGS. 6A and 6B), demonstrating the accuracy of the m⁶A-sac-seq method.

### Example 4—Klentaq Enzyme Generates Mutations at m⁶A Sites

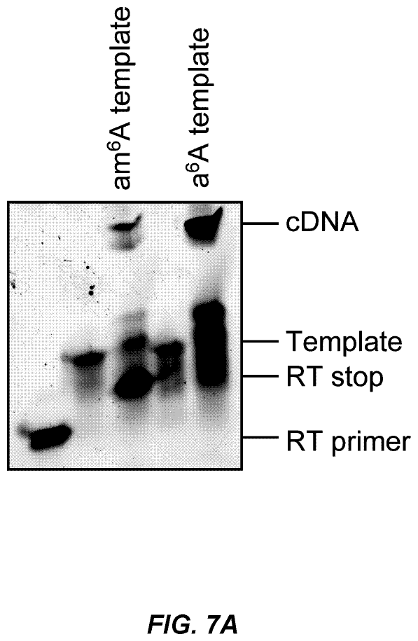

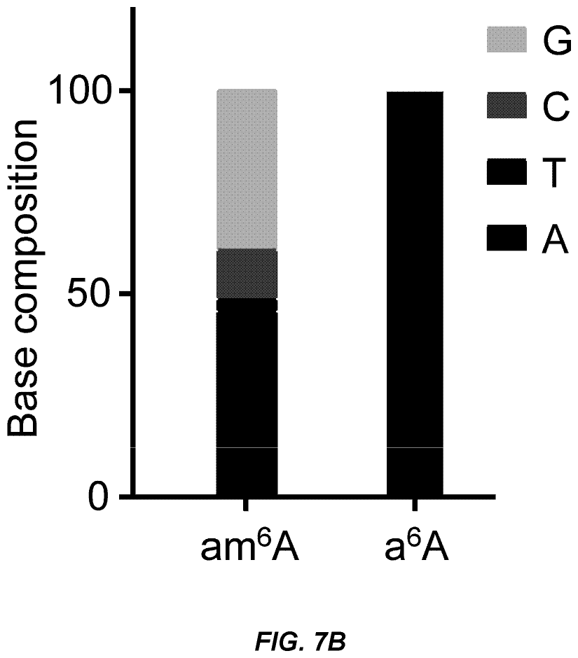

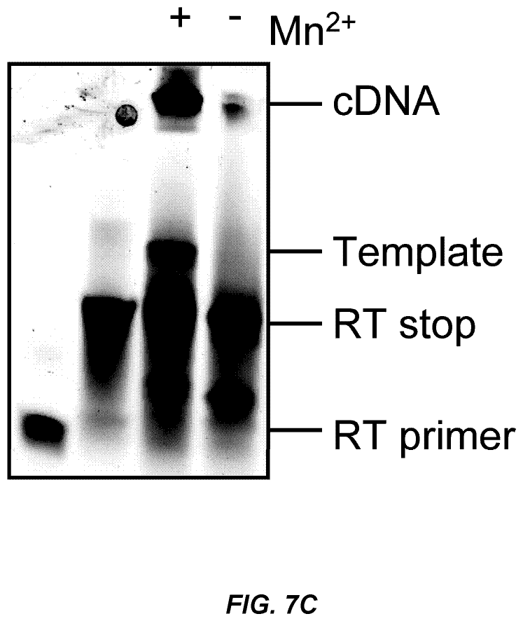

Wild-type Klentaq was used to induce reverse transcription using an am⁶A-containing template and an a⁶A-containing template. FIG. 7A shows that readthrough efficiency of the wild-type Klentaq enzyme was only about 10%. FIG. 7B shows that am⁶A induced about 50% misincorporation (i.e., mutation) with wild type Klentaq enzyme during reverse transcription, while a⁶A gave close to background mutation level. Mn²⁺ was provided and reverse transcription of the templates performed. FIG. 7C shows that the addition of Mn²⁺ increases readthrough efficiency to about 90%.

### Example 5—Klentaq Enzyme Directed Evolution

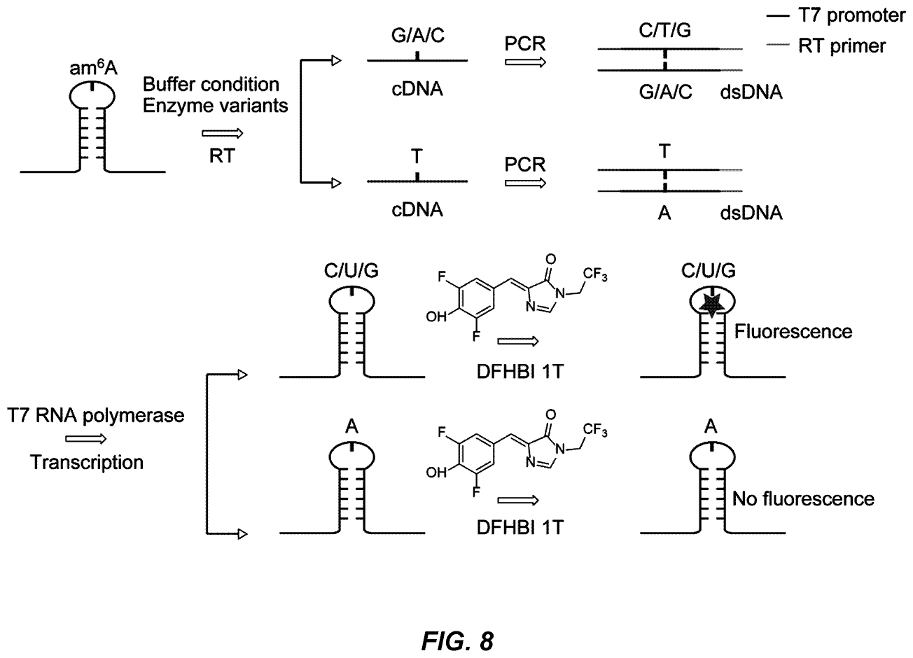

Wild-type Klentaq was subjected to directed evolution as outlined in FIG. 8. Broccoli, an RNA aptamer that binds and activates fluorescence of DFHB1 and shows robust green fluorescence, was engineered at several sites by replacing them with am⁶A. Only when Klentaq variants induce misincorporations under optimal reverse transcription buffer conditions could this engineered Broccoli bind DFHB 1T and emit green fluorescence.

### Example 6—Detection of N⁶-Methyladenosine with m⁶A-Sac-Seq Using a Modified Klentaq Enzyme

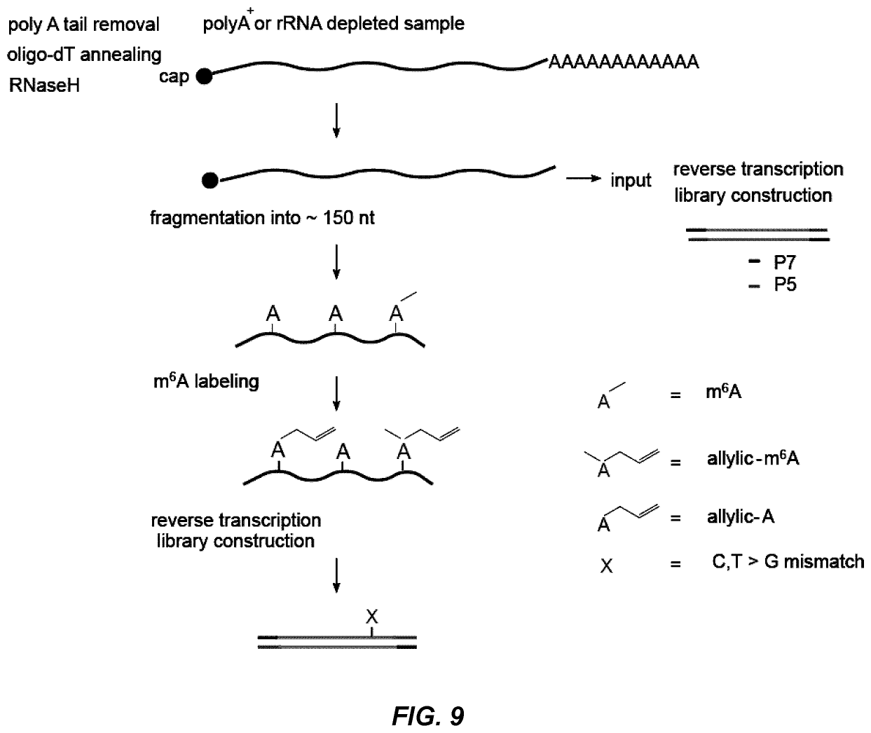

An overview of another example m⁶A-sac-seq method using a modified Klentaq enzyme is shown in FIG. 9 and is performed as follows:
i) Purified RNA is annealed with oligo-dT, digested with Rnase H to remove poly(A) tail followed by fragmentation into polynucleotide fragments with length of about 150 bp.
ii) Fragmented RNA sample is evenly divided into 2 parts: reference input group and experimental group. Reference input group is processed through library construction free of extra treatment.
iii) RNA in the experimental group is subjected to allyl labeling by recombinant MjDim1 and allyl-SAM cofactor under optimized conditions.
iv) cDNA is synthesized using the evolved Klentaq enzyme that directly reads am⁶A sites as mutations, with a⁶A sites giving close to background mutation rate.
v) A comparison of results from treated RNA versus reference input identifies m⁶A sites in the transcriptome. Calibration probes with gradient m⁶A levels are used to identify the modification fraction information at each modified site.

### Example 7—Detection of N⁶-Methyladenosine with m⁶A-Sac-Seq Using a Bst Enzyme

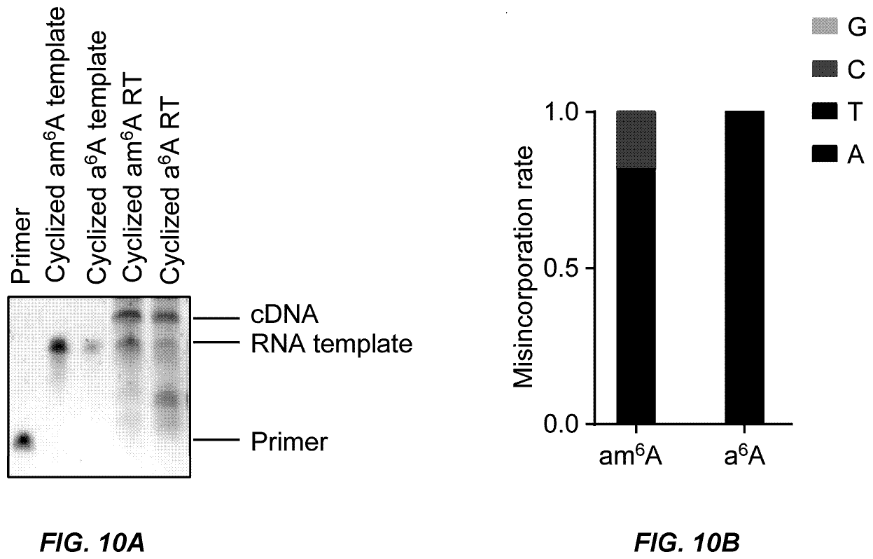

Bst 2.0 enzyme was used to induce reverse transcription using a cyclized am6A (ethanoadenine-m⁶A)-containing template and a cyclized a⁶A (N¹, N⁶-ethanoadenine)-containing template. FIG. 10A shows that readthrough efficiency of the Bst 2.0 enzyme was about 100%. FIG. 10B shows that cyclized am⁶A induced about 80% misincorporation (i.e., mutation) with Bst2.0 enzyme during reverse transcription, while cyclized a⁶A gave close to background mutation level.

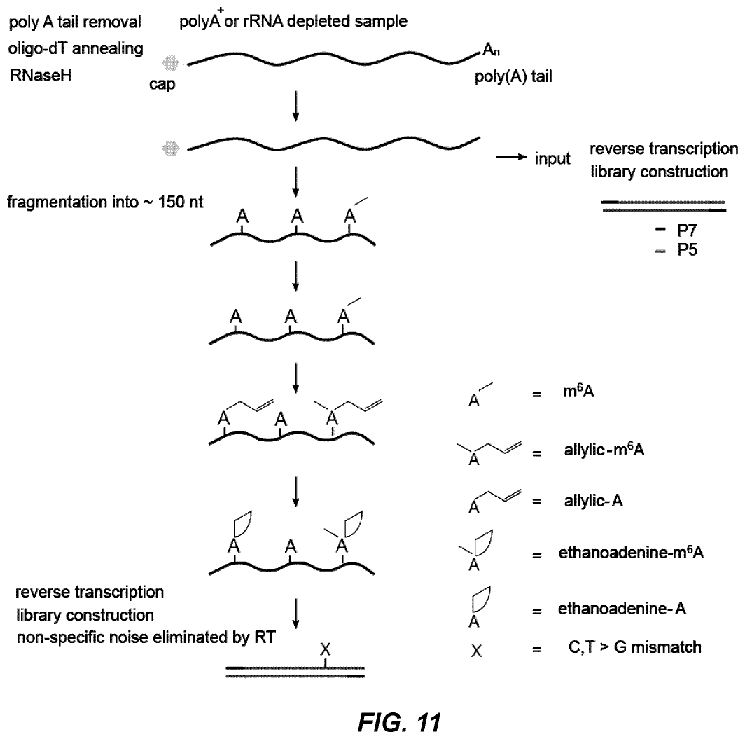

An overview of another example m⁶A-sac-seq method using a Bst enzyme (e.g., Bst 2.0 or Bst 3.0) is shown in FIG. 11 and is performed as follows:
i) Purified RNA is annealed with oligo-dT, digested with Rnase H to remove poly(A) tail followed by fragmentation into ˜150 nucleotides.
ii) Fragmented RNA sample is evenly divided into 2 parts: reference input group and experimental group. Reference input group will be processed through library construction free of extra treatment.
iii) RNA in the experimental group will be subject to allyl labeling by recombinant MjDim1 and allylic-SAM cofactor under optimized conditions, followed by 12 induced cyclization.
iv) cDNA is synthesized using the Bst enzyme that directly reads am⁶A sites as mutations, with a⁶A sites giving close to background mutation rate.
v) A comparison of results from treated RNA versus reference input identify m⁶A sites in the transcriptome. Using calibration probes with gradient m⁶A level can yield the modification fraction information at each modified site.

### Example 8—Bst 2.0 Induces Mutation Specifically on m6A Sites and Distinguishes Between Methylated and Unmethylated Sites

### Results

To assess the mutation effects on N⁶-allyl N⁶-methyladenosine (am⁶A) using Bst 2.0, synthetic RNA probes with either NNm⁶ANN or NNam⁶ANN motifs were subjected to m⁶A-sac-seq protocol and subsequent high-throughput sequencing. N represents evenly distributed random nucleotides, thus including 256 different motifs in each set of probes. For NNm⁶ANN probes, pre-mixed, uniquely barcoded probes that contained m⁶A at 0%, 25%, 50%, 75% and 100% modification levels, respectively, were used. These probes were subjected to allyl transfer using Mjdim1 as in the standard m⁶A-sac-seq (described in Example 2), I₂-induced cyclization and then followed by reverse transcription using BST 2.0. NNam⁶ANN probes with chemically synthesized allylated m⁶A (am⁶A) in the middle were used as the control without Mjdim1 treatment. To further assess the background mutation from N⁶-allyladenosine (a⁶A) generated by non-specific activity of Mjdim1 on unmethylated A, half of the NNm⁶ANN probes were demethylated by FTO as is described in Example 2 prior to Mjdim1 treatment and cyclization as controls.

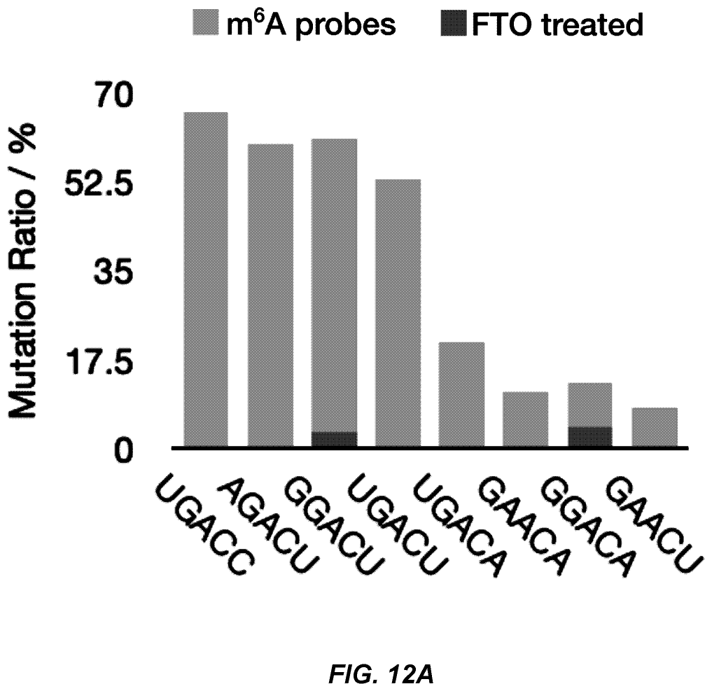

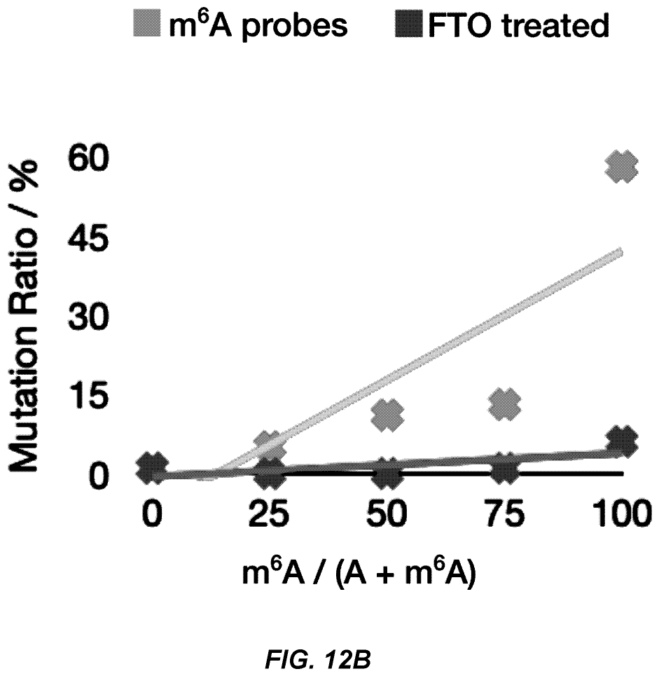

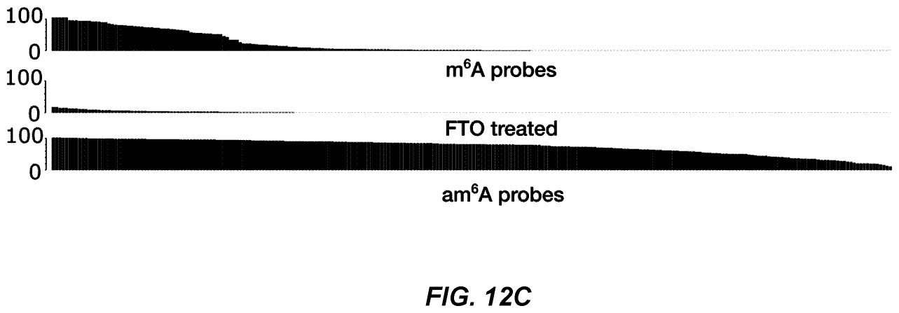

When probes with 100% m⁶A modification level were used, higher than 50% mutation rate could be achieved on 4 DRACH m⁶A consensus motifs including the most abundant AGACU/GGACU, with minimal background (less than 5%) (FIG. 12A). Linear regression of GGACU motif mutations on probes with different m⁶A fractions showed moderate linearity, with consistently low background mutations (FIG. 12B). Further, the NNm⁶ANN probes with and without demethylation by FTO were subjected to Mjdim1 treatment, I₂-induced cyclization, followed by reverse transcription using BST 2.0. All 256 motifs without demethylation showed notable mutations, whereas very low mutations were observed for the same probes with m⁶A demethylated by FTO (FIG. 12C). Lastly, pre-allylated am⁶A probes already containing the allyl group on m⁶A at 100% level generated high mutation rate throughout all motifs and >25% mutation on all DRACH motifs, showing that Bst 2.0 enzyme works effectively on all sequence contexts.

These results demonstrated that Bst 2.0 is capable of generating mutation rates comparable to that generated by HIV RT in standard m⁶A-sac-seq procedure (e.g., as described in Example 2). Most importantly, this enzyme generated very low mutation on unmethylated A sites, eliminating background mutations. The use of Bst 2.0 can eliminate the need to use demethylation control (e.g., FTO treatment) in m⁶A-sac-seq.

### Methods

Bst reverse transcription was carried out on beads by mixing 50 ng of biotinylated RNA immobilized on 20 μL Dynabeads MyOne Streptavidin C1 (ThermoFisher) with 1 μL of 2 μM sequence-specific primer. After denaturation at 65° C. for 2 min, add 4.5 μL of 10× isothermal amplification buffer (NEB), 4.5 μL of 10 mM dNTP (ThermoFisher), 2.7 μL of 100 mM MgSO₄ (NEB), 1.1 μL of RNaseOUT (ThermoFisher), 7.2 μL of 50% PEG 4000 (Rigaku) and 5 μL of 120 U/μL Bst 2.0 (M0537M, NEB). After mixing well the reaction was incubated at 37° C. for 3 hours. Then the beads were rinsed with 50 μL of 10 mM Tris-HCl pH 7.5 and resuspended in 17 μL of RNase-free water. After addition of 2 μL of 10× RNase H buffer (NEB) and 1 μL of RNase H (NEB) the reaction was further incubation at 37° C. for 20 min. The product cDNA was purified by rinsing the beads sequentially with 50 μL of 0.1% (v/v) Tween 20 in 1×PBS, 50 μL of Binding/Wash buffer (10 mM Tris-HCl pH 7.5, 1 mM EDTA, 2 M NaCl), and twice with 50 μL of 10 mM Tris-HCl pH 7.5. RNA was released by boiling the beads in 50 μL of RNase-free water at 95° C. for 10 min. The resulting cDNA could be used for downstream NGS library construction.

The probes used were as follows:

0% NNm⁶ANN probe-
UAUCUGUCUCGACGUNN(m⁶A)NNGGCCUUUGCAACUAGAAUUACACCAU
AAUUGCU;
25% NNm⁶ANN probe-
UAUCUGUCUCGACGUNN(m⁶A)NNGGCAUUCAAGCCUAGAAUUACACCAU
AAUUGCU;
50% NNm⁶ANN probe-
UAUCUGUCUCGACGUNN(m⁶A)NNGGCGAGGUGAUCUAGAAUUACACCAU
AAUUGCU;
75% NNm⁶ANN probe-
UAUCUGUCUCGACGUNN(m⁶A)NNGGCUUCAACAACUAGAAUUACACCAU
AAUUGCU;
100% NNm⁶ANN probe-
UAUCUGUCUCGACGUNN(m⁶A)NNGGCGAUGGUUUCUAGAAUUACACCAU
AAUUGCU.

All (m⁶A) sites could be substituted to A and then the two could be mixed at desired modification fractions.

NNam6ANN probe:
UCGACGUNN(am⁶A)NNGGCATTGCT.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Frye, M., Harada, B. T., Behm, M. & He, C. RNA modifications modulate gene expression during development. Science 361, 1346-1349, doi:10.1126/science.aau1646 (2018).
2. Roundtree, I. A., Evans, M. E., Pan, T. & He, C. Dynamic RNA Modifications in Gene Expression Regulation. Cell 169, 1187-1200, doi:10.1016/j.cell.2017.05.045 (2017).
3. O'Farrell, H. C., Musayev, F. N., Scarsdale, J. N. & Rife, J. P. Binding of adenosine-based ligands to the MjDim1 rRNA methyltransferase: implications for reaction mechanism and drug design. Biochemistry 49, 2697-2704, doi:10.1021/bi901875x (2010).
4. O'Farrell, H. C., Pulicherla, N., Desai, P. M. & Rife, J. P. Recognition of a complex substrate by the KsgA/Dim1 family of enzymes has been conserved throughout evolution. Rna 12, 725-733, doi:10.1261/rna.2310406 (2006).
5. Zhang, J. & Zheng, Y. G. SAM/SAH Analogs as Versatile Tools for SAM-Dependent Methyltransferases. ACS chemical biology 11, 583-597, doi:10.1021/acschembio.5b00812 (2016).
6. Shu, X. et al. N(6)-Allyladenosine: A New Small Molecule for RNA Labeling Identified by Mutation Assay. Journal of the American Chemical Society 139, 17213-17216, doi:10.1021/jacs.7b06837 (2017).
7. Dominissini, D. et al. Topology of the human and mouse m6A RNA methylomes revealed by m6A-Seq. Nature 485, 201-206, doi:10.1038/nature11112 (2012).
8. Xiao, Y. et al. An Elongation- and Ligation-Based qPCR Amplification Method for the Radiolabeling-Free Detection of Locus-Specific N(6)-Methyladenosine Modification. Angewandte Chemie 57, 15995-16000, doi:10.1002/anie.201807942 (2018).
9. Aschenbrenner, J. et al. Engineering of a DNA Polymerase for Direct m(6)A Sequencing. Angewandte Chemie 57, 417-421, doi:10.1002/anie.201710209 (2018).

1. A method for detecting a methylated nucleotide of a nucleic acid molecule comprising:
(a) incubating the nucleic acid molecule with a methyltransferase enzyme and a S-adenosyl-1-methionine (SAM) analog comprising a functional group under conditions sufficient to attach the functional group to the methylated nucleotide;
(b) subjecting the nucleic acid molecule to conditions sufficient to generate a complementary nucleic acid molecule comprising a mutation at a residue corresponding to the methylated nucleotide; and
(c) sequencing the complementary nucleic acid molecule.

2. The method of claim 1, wherein the methylated nucleotide is a methylated adenosine.

3. The method of claim 2, wherein the methylated nucleotide is N⁶-methyladenosine.

4. The method of claim any of claims 1-3, wherein the functional group is attached to a sulfur atom of the SAM analog.

5. The method of claim 4, wherein the SAM analog has formula: 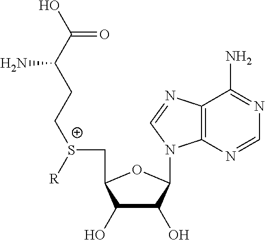
wherein R comprises the functional group.

6. The method of any of claims 1-5, wherein the functional group is not a methyl group.

7. The method of any of claims 1-6, wherein the functional group has at least two carbon atoms.

8. The method of claim 7, wherein the functional group is an alkyl group having at least two carbons or an olefinic group having at least two carbons.

9. The method of claim 8, wherein the functional group is an allyl group.

10. The method of claim 9, wherein the SAM analog has formula:

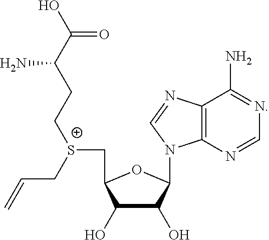

11. The method of any of claims 1-10, wherein the methyltransferase is capable of preferentially attaching the functional group to a methylated nucleotide relative to an unmethylated nucleotide under appropriate conditions.

12. The method of any of claims 1-11, wherein the methyltransferase is an RNA methyltransferase.

13. The method of claim 12, wherein the RNA methyltransferase is a dimethyltransferase.

14. The method of claim 13, wherein the dimethyltransferase is a Dim1/KsgA dimethyltransferase.

15. The method of claim 14, wherein the dimethyltransferase is Dim1 or KsgA.

16. The method of claim 15, wherein the dimethyltransferase is HsDim1, ScDim1, or MjDim1.

17. The method of claim 16, wherein the dimethyltransferase is MjDim1.

18. The method of any of claims 1-17, further comprising incubating the nucleic acid molecule with a diatomic halogen molecule.

19. The method of claim 18, wherein incubating the nucleic acid molecule with the diatomic halogen molecule attaches a halogen atom from the diatomic halogen molecule to the nucleotide.

20. The method of claim 19, wherein the diatomic halogen molecule is iodine (I₂).

21. The method of any of claims 1-20, wherein (b) comprises subjecting the nucleic acid molecule to a reverse transcription reaction with a reverse transcriptase (RT) to generate the complementary nucleic acid molecule.

22. The method of claim 21, wherein the complementary nucleic acid molecule is a cDNA molecule.

23. The method of claim 21 or 22, wherein the RT is an HIV RT or variant thereof, an M-MuLV RT or variant thereof, an AMV RT or variant thereof, a Klentaq polymerase or variant thereof, or a Bst polymerase or variant thereof.

24. The method of claim 23, wherein the RT is a Bst polymerase or variant thereof.

25. The method of claim 24, wherein the RT is Bst 2.0 DNA polymerase.

26. The method of claim 23, wherein the RT is a Klentaq polymerase or variant thereof.

27. The method of any of claims 1-26, wherein the sequencing comprises next generation sequencing.

28. The method of any of claims 1-26, wherein the sequencing comprises single molecule sequencing.

29. The method of any of claims 1-26, wherein the sequencing comprises nanopore sequencing.

30. The method of any of claims 1-29, wherein the methylated nucleotide is a methylated adenosine, and wherein the residue does not comprise an adenine.

31. The method of any of claims 1-30, wherein the methylated nucleotide is a methylated adenosine, and wherein the residue comprises a guanine, a thymine, or a cytosine.

32. The method of any of claims 1-31, further comprising identifying the mutation in the additional nucleic acid molecule as corresponding to the methylated nucleotide.

33. The method of any of claims 1-32, wherein the nucleic acid molecule is a ribonucleic acid molecule.

34. The method of claim 33, wherein the ribonucleic acid molecule is a messenger RNA (mRNA) molecule.

35. The method of claim 34, further comprising, prior to (a), providing an oligo-dT primer to the mRNA molecule to generate a double stranded region.

36. The method of claim 35, further comprising providing a nuclease and subjecting the mRNA to conditions sufficient to digest the double stranded region with the nuclease.

37. The method of claim 36, wherein the nuclease is RNase H.

38. The method of any of claims 1-37, wherein the nucleic acid molecule is a fragment of a longer nucleic acid.

39. The method of claim 38, wherein the fragment is between 100 and 200 nucleotides in length.

40. The method of any of claims 1-39, wherein the nucleic acid molecule is isolated from a sample from a subject.

41. The method of claim 40, wherein the nucleic acid molecule is isolated from a biopsy sample.

42. The method of claim 40 or 41, wherein the sample is a liquid sample.

43. The method of any of claims 1-42, wherein the nucleic acid molecule is isolated from a vesicle.

44. The method of claim 43, wherein the vesicle is an exosome.

45. The method of any of claims 1-42, wherein the nucleic acid molecule is a cell free nucleic acid molecule.

46. The method of claim 45, wherein the cell free nucleic acid molecule is a cell free RNA (cfRNA) molecule.

47. A kit comprising:
(a) a S-adenosyl-1-methionine (SAM) analog comprising a functional group; and
(b) a dimethyltransferase.

48. The kit of claim 47, wherein the dimethyltransferase is capable of preferentially attaching the functional group to a methylated nucleotide relative to an unmethylated nucleotide under appropriate conditions.

49. The kit of claim 47 or 48, wherein the dimethytransferase is an RNA methyltransferase.

50. The kit of claim 49, wherein the dimethyltransferase is a Dim1/KsgA dimethyltransferase.

51. The kit of claim 50, wherein the dimethyltransferase is Dim1 or KsgA.

52. The kit of claim 51, wherein the dimethyltransferase is HsDim1, ScDim1, or MjDim1.

53. The kit of claim 52, wherein the dimethyltransferase is MjDim1.

54. The kit of any of claims 47-53, wherein the functional group is attached to a sulfur atom of the SAM analog.

55. The kit of claim 54, wherein the SAM analog has formula: 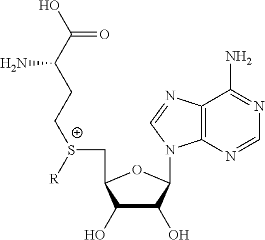
wherein R comprises the functional group.

56. The kit of any of claims 47-55, wherein the functional group is not a methyl group.

57. The kit of any of claims 47-56, wherein the functional group has at least two carbon atoms.

58. The kit of claim 57, wherein the functional group is an alkyl group having at least two carbons or an olefinic group having at least two carbons.

59. The kit of claim 58, wherein the functional group is an allyl group.

60. The kit of claim 59, wherein the SAM analog has formula:

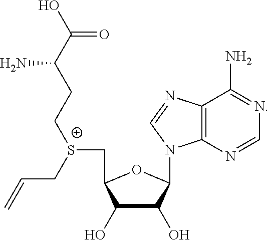

61. The kit of any of claims 47-60, further comprising a oligo-dT primer.

62. The kit of any of claims 47-61, further comprising a nuclease.

63. The kit of claim 62, wherein the nuclease is RNase H.

64. The kit of any of claims 47-63, further comprising a reverse transcriptase (RT).

65. The kit of claim 64, wherein the RT is an HIV RT or variant thereof, an M-MuLV RT or variant thereof, an AMV RT or variant thereof, a Bst polymerase or variant thereof, or a Klentaq polymerase or variant thereof.

66. The kit of claim 65, wherein the RT is a Bst polymerase or variant thereof.

67. The kit of claim 66, wherein the RT is Bst 2.0 DNA polymerase.

68. The kit of claim 65, wherein the RT is a Klentaq polymerase or variant thereof.

69. The kit of any of claims 47-68, further comprising an RNA demethylase.

70. The kit of claim 69, wherein the RNA demethylase is fat mass and obesity-associated protein (FTO).

71. The kit of any of claims 47-70, further comprising a manganese salt.

72. The kit of any of claims 47-71, further comprising dNTPs.

73. The kit of any of claims 47-72, further comprising nuclease-free water.

74. A method for analyzing a methylated messenger ribonucleic acid (mRNA) molecule comprising an N⁶-methyladenosine, the method comprising:
(a) fragmenting the mRNA molecule to generate a fragment comprising the N⁶-methyladenosine;
(b) providing a methyltransferase and a S-adenosyl-1-methionine (SAM) analog comprising an allyl group under conditions sufficient to attach the allyl group to the N⁶-methyladenosine in the fragment;
(c) incubating the fragment with a reverse transcriptase under conditions sufficient to generate a cDNA molecule comprising a residue corresponding to the N⁶-methyladenosine, wherein the residue comprises a guanine, thymine, or cytosine;
(d) sequencing the cDNA molecule to generate a sequence; and
(e) identifying a location of the N⁶-methyladenosine in the mRNA molecule using the sequence.

75. The method of claim 74, further comprising, prior to (a), incubating the mRNA molecule with an oligo-dT primer under conditions sufficient to hybridize the oligo-dT primer to a region of the mRNA molecule, thereby generating a double stranded region.

76. The method of claim 75, further comprising providing a nuclease under conditions sufficient to digest the double stranded region.

77. The method of claim 76, wherein the nuclease is RNase H.

78. The method of any of claims 74-77, wherein the SAM analog has formula:

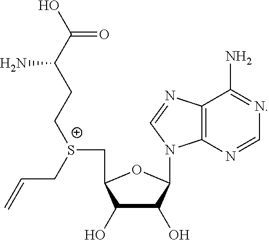

79. The method of any of claims 74-78, wherein the methyltransferase is an RNA methyltransferase.

80. The method of claim 79, wherein the RNA methyltransferase is a dimethyltransferase.

81. The method of claim 80, wherein the dimethyltransferase is a Dim1/KsgA dimethyltransferase.

82. The method of claim 81, wherein the dimethyltransferase is Dim1 or KsgA.

83. The method of claim 82, wherein the dimethyltransferase is HsDim1, ScDim1, or MjDim1.

84. The method of claim 83, wherein the dimethyltransferase is MjDim1.

85. The method of any of claims 74-84, further comprising, subsequent to (b), incubating the mRNA molecule with a diatomic halogen molecule.

86. The method of claim 85, wherein incubating the mRNA molecule with the diatomic halogen molecule attaches a halogen atom from the diatomic halogen molecule to the nucleotide.

87. The method of claim 85 or 86, wherein the diatomic halogen molecule is iodine (I₂).

88. The method of any of claims 74-87, wherein the reverse transcriptase (RT) is an HIV RT or variant thereof, an M-MuLV RT or variant thereof, an AMV RT or variant thereof, a Bst polymerase or variant thereof, or a Klentaq polymerase or variant thereof.

89. The method of claim 88, wherein the RT is a Bst polymerase or variant thereof.

90. The method of claim 89, wherein the RT is Bst 2.0 DNA polymerase.

91. The method of claim 88, wherein the RT is a Klentaq polymerase or variant thereof.

92. The method any of claims 74-91, wherein the fragment is between 100 and 200 nucleotides in length.

93. The method any of claims 74-92, wherein the mRNA molecule is isolated from a sample from a subject.

94. The method claim 93, wherein the mRNA molecule is isolated from a biopsy sample.

95. The method claim 93 or 94, wherein the sample is a liquid sample.

96. The method of any of claims 74-95, wherein the mRNA molecule is isolated from a vesicle.

97. The method of claim 96, wherein the vesicle is an exosome.

98. The method any of claims 74-84, wherein the mRNA molecule is a cell free ribonucleic acid (cfRNA) molecule.

99. A method for modifying a nitrogenous base methylated at a nitrogen atom comprising:
(a) providing a methyltransferase enzyme and a S-adenosyl-1-methionine (SAM) analog comprising a functional group; and
(b) subjecting the methyltransferase enzyme and the SAM analog to conditions sufficient to attach the functional group to the nitrogen atom.

100. The method of claim 99, wherein the nitrogenous base is a nitrogenous base of a nucleoside.

101. The method of claim 100, wherein the nitrogenous base is a nitrogenous base of a nucleotide.

102. The method of claim 101, wherein the nucleotide is a nucleotide of a ribonucleic acid (RNA).

103. The method of claim 102, wherein the nucleotide is a methylated adenosine.

104. The method of claim 103, wherein the nucleotide is N⁶-methyladenosine.

105. The method of any of claims 99-104, further comprising incubating the nitrogenous base with a diatomic halogen molecule.

106. The method of claim 105, wherein the diatomic halogen molecule is iodine (I₂).

107. The method of any of claims 99-106, wherein the methyltransferase is capable of preferentially attaching the functional group to a methylated nucleotide relative to an unmethylated nucleotide under appropriate conditions.

108. The method of any of claims 99-107, wherein the methyltransferase is an RNA methyltransferase.

109. The method of claim 108, wherein the RNA methyltransferase is a dimethyltransferase.

110. The method of claim 109, wherein the dimethyltransferase is a Dim1/KsgA dimethyltransferase.

111. The method of claim 110, wherein the dimethyltransferase is Dim1 or KsgA.

112. The method of claim 111, wherein the dimethyltransferase is HsDim1, ScDim1, or MjDim1.

113. The method of claim 112, wherein the dimethyltransferase is MjDim1.

114. The method of any of claims 99-113, wherein the functional group is attached to a sulfur atom of the SAM analog.

115. The method of claim 114, wherein the SAM analog has formula: 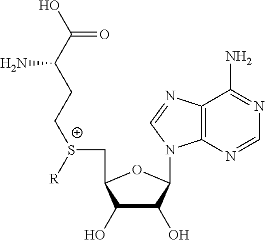
wherein R comprises the functional group.

116. The method of any of claims 99-115, wherein the functional group is not a methyl group.

117. The method of any of claims 99-116, wherein the functional group has at least two carbon atoms.

118. The method of claim 117, wherein the functional group is an alkyl group having at least two carbons or an olefinic group having at least two carbons.

119. The method of claim 118, wherein the functional group is an allyl group.

120. The method of claim 119, wherein the SAM analog has formula:

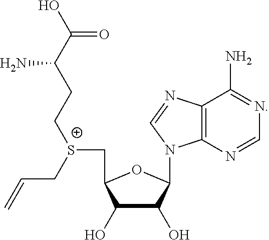

121. A method for detecting a methylated nucleotide in a ribonucleic acid comprising:
(a) attaching a functional group to a nitrogen atom on the nucleotide;
(b) generating, from the ribonucleic acid, a complementary nucleic acid comprising a mutation at a residue corresponding to the nucleotide; and
(c) sequencing the complementary nucleic acid.

122. The method of claim 121, wherein the nucleotide is a methylated adenosine.

123. The method of claim 122, wherein the nucleotide is N⁶-methyladenosine.

124. The method of any of claims 121-123, wherein (a) comprises providing a S-adenosyl-1-methionine (SAM) analog comprising the functional group.

125. The method of claim 124, wherein the functional group is attached to a sulfur atom of the SAM analog.

126. The method of claim 125, wherein the SAM analog has formula: 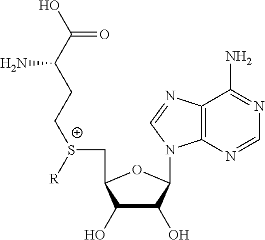
wherein R comprises the functional group.

127. The method of any of claims 121-126, wherein the functional group is not a methyl group.

128. The method of any of claims 121-127, wherein the functional group has at least two carbon atoms.

129. The method of claim 128, wherein the functional group is an alkyl group having at least two carbons or an olefinic group having at least two carbons.

130. The method of claim 129, wherein the functional group is an allyl group.

131. The method of claim 130, wherein the SAM analog has formula:

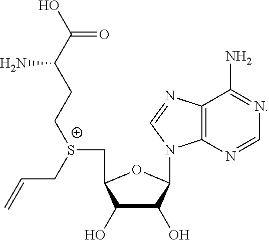

132. The method of any of claims 121-131, wherein (a) comprises attaching the functional group with a methyltransferase.

133. The method of claim 132, wherein the methyltransferase is capable of preferentially attaching the functional group to a methylated nucleotide relative to an unmethylated nucleotide under appropriate conditions.

134. The method of claim 132 or 133, wherein the methyltransferase is an RNA methyltransferase.

135. The method of claim 134, wherein the RNA methyltransferase is a dimethyltransferase.

136. The method of claim 135, wherein the dimethyltransferase is a Dim1/KsgA dimethyltransferase.

137. The method of claim 136, wherein the dimethyltransferase is Dim1 or KsgA.

138. The method of claim 137, wherein the dimethyltransferase is HsDim1, ScDim1, or MjDim1.

139. The method of claim 138, wherein the dimethyltransferase is MjDim1.

140. The method of any of claims 121-139, further comprising incubating the ribonucleic acid with a diatomic halogen molecule.

141. The method of claim 140, wherein incubating the ribonucleic acid with the diatomic halogen molecule attaches a halogen atom from the diatomic halogen molecule to the nucleotide.

142. The method of claim 141, wherein the diatomic halogen molecule is iodine (I₂).

143. The method of any of claims 121-142, wherein (b) comprises performing a reverse transcription reaction with a reverse transcriptase (RT).

144. The method of claim 143, wherein the RT is an HIV RT or variant thereof, an M-MuLV RT or variant thereof, an AMV RT or variant thereof, a Bst polymerase or variant thereof, or a Klentaq polymerase or variant thereof.

145. The method of claim 144, wherein the RT is a Bst polymerase or variant thereof.

146. The method of claim 145, wherein the RT is Bst 2.0 DNA polymerase.

147. The method of claim 144, wherein the RT is a Klentaq polymerase or variant thereof.

148. The method of any of claims 121-147, wherein the sequencing comprises next generation sequencing.

149. The method of any of claims 121-147, wherein the sequencing comprises single molecule sequencing.

150. The method of any of claims 121-147, wherein the sequencing comprises nanopore sequencing.

151. The method of any of claims 121-150, wherein the nucleotide is adenosine, and wherein the residue does not comprise an adenine.

152. The method of any of claims 121-150, wherein the nucleotide is adenosine, and wherein the residue comprises a guanine, a thymine, or a cytosine.

153. The method of any of claims 121-152, further comprising identifying the mutation in the complementary nucleic acid as corresponding to the nucleotide in the ribonucleic acid.

154. The method of any of claims 121-153, wherein the ribonucleic acid is messenger RNA (mRNA).

155. The method of claim 154, further comprising, prior to (a), annealing an oligo-dT primer to the mRNA to generate a double-stranded region.

156. The method of claim 155, further comprising digesting the double stranded region with a nuclease.

157. The method of claim 156, wherein the nuclease is RNase H.

158. The method of any of claims 121-157, further comprising, prior to (a), generating complementary deoxyribonucleic acid (cDNA) from the ribonucleic acid and sequencing the cDNA.

159. The method of any of claims 121-158, wherein the ribonucleic acid is a fragment of a longer ribonucleic acid.

160. The method of claim 159, wherein the fragment is between 100 and 200 nucleotides in length.

161. The method of any of claims 121-160, wherein the ribonucleic acid is isolated from a sample from a subject.

162. The method of claim 161, wherein the ribonucleic acid is isolated from a biopsy sample.

163. The method of claim 161 or 162, wherein the sample is a liquid sample.

164. The method of any of claims 121-163, wherein the nucleic acid molecule is isolated from a vesicle.

165. The method of claim 164, wherein the vesicle is an exosome.

166. The method of any of claims 121-163, wherein the ribonucleic acid is a cell free ribonucleic acid (cfRNA).


SEQUENCE LISTING (11 sequences)

SEQ ID NO: 1 - RNA, 53 nt, Artificial Sequence
  Description of Artificial Sequence Synthetic probe
  modified_base (16)..(17): a, c, u, g, unknown or other
  modified_base (19)..(20): a, c, u, g, unknown or other
uaucugucucgacgunnannggccuuugcaacuagaauuacaccauaauugcu

SEQ ID NO: 2 - RNA, 53 nt, Artificial Sequence
  Description of Artificial Sequence Synthetic probe
  modified_base (16)..(17): a, c, u, g, unknown or other
  modified_base (19)..(20): a, c, u, g, unknown or other
uaucugucucgacgunnannggcauucaagccuagaauuacaccauaauugcu

SEQ ID NO: 3 - RNA, 53 nt, Artificial Sequence
  Description of Artificial Sequence Synthetic probe
  modified_base (16)..(17): a, c, u, g, unknown or other
  modified_base (19)..(20): a, c, u, g, unknown or other
uaucugucucgacgunnannggcgaggugaucuagaauuacaccauaauugcu

SEQ ID NO: 4 - RNA, 53 nt, Artificial Sequence
  Description of Artificial Sequence Synthetic probe
  modified_base (16)..(17): a, c, u, g, unknown or other
  modified_base (19)..(20): a, c, u, g, unknown or other
uaucugucucgacgunnannggcuucaacaacuagaauuacaccauaauugcu

SEQ ID NO: 5 - RNA, 53 nt, Artificial Sequence
  Description of Artificial Sequence Synthetic probe
  modified_base (16)..(17): a, c, u, g, unknown or other
  modified_base (19)..(20): a, c, u, g, unknown or other
uaucugucucgacgunnannggcgaugguuucuagaauuacaccauaauugcu

SEQ ID NO: 6 - DNA, 21 bp, Artificial Sequence
  Description of Artificial Sequence Synthetic probe
  Description of Combined DNA/RNA Molecule Synthetic probe
  modified_base (8)..(9): a, c, t, g, unknown or other
  modified_base (11)..(12): a, c, t, g, unknown or other
ucgacgunnannggcattgct

SEQ ID NO: 7 - RNA, 12 nt, Artificial Sequence
  Description of Artificial Sequence Synthetic oligonucleotide
cguggacuggcu

SEQ ID NO: 8 - RNA, 12 nt, Artificial Sequence
  Description of Artificial Sequence Synthetic oligonucleotide
cguggacuggcu

SEQ ID NO: 9 - RNA, 12 nt, Artificial Sequence
  Description of Artificial Sequence Synthetic oligonucleotide
cguggacuggcu

SEQ ID NO: 10 - DNA, 20 bp, Artificial Sequence
  Description of Artificial Sequence Synthetic oligonucleotide
tcgacgtggdctggcattgc

SEQ ID NO: 11 - RNA, 12 nt, Artificial Sequence
  Description of Artificial Sequence Synthetic oligonucleotide
aaaaaaaaaaaa